(19) United States
(12) Patent Application Publication
(10) Pub. No.: US 2016/0237066 A1
(43) Pub. Date: Aug. 18, 2016

(54) IMPROVED PROCESS FOR THE PREPARATION OF ((3S,5R)-5-((1H-1,2,4-TRIAZOL-1-YL)METHYL)-5-(2,4-DIFLUOROPHENYL)TETRAHYDROFURAN-3-YL)METHYL-4-METHYLBENZENESULFONATE

(71) Applicant: MSN LABORATORIES PRIVATE LIMITED, Hyderabad (IN)

(72) Inventors: Srinivasan Thirumalai Rajan, Hyderabad (IN); Sajja Eswaraiah, Hyderabad (IN); Maramreddy Sahadeva Reddy, Hyderabad (IN)

(73) Assignee: MSN LABORATORIES PRIVATE LIMITED, Hyderabad (IN)

(21) Appl. No.: 15/031,536

(22) PCT Filed: Oct. 21, 2014
(86) PCT No.: PCT/IN2014/000673

§ 371 Date: Apr. 22, 2016

(30) Foreign Application Priority Data
Oct. 22, 2013 (IN) 4757/CHE/2013

(51) Int. Cl. C07D 405/06; C07D 405/14; C07D 413/06; C07D 307/12; C07D 263/26

(52) U.S. Cl. CPC C07D 405/06; C07D 307/12; C07D 263/26; C07D 413/06; C07D 405/14

(57) ABSTRACT

The present invention relates to process for the preperation of (3S,5R)-5-((1H-1,2,4-triazol-1-yl)methyl)-5-(2,4-difluorophenyl)tetrahydrofuran-3-yl)methyl-4-methylbenzene sulfonate compound of formula-1 through novel intermediates. Further the said compound of formula-1 is useful as a key intermediate for the preparation of Posaconazole.

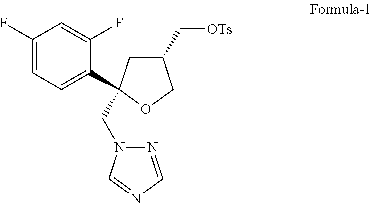 [SMILES: Cc1ccc(S(=O)(=O)OC[C@@H]2CO[C@@](Cn3cncn3)(c3ccc(F)cc3F)C2)cc1]

### RELATED APPLICATION

This application claims the benefit of priority of our Indian patent application number 4757/CHE/2013 filed on Oct. 22, 2013 which is incorporated herein by reference.

### FIELD OF THE INVENTION

The present invention relates to an improved process for the preparation of ((3S,5R)-5-((1H-1,2,4-triazol-1-yl)methyl)-5-(2,4-difluorophenyl)tetrahydrofuran-3-yl)methyl-4-methyl benzene sulfonate compound of formula-1 through novel intermediates. Further the said compound of formula-1 is useful as a key intermediate for the preparation of Posaconazole.

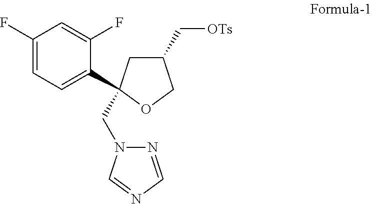 [SMILES: Cc1ccc(S(=O)(=O)OC[C@@H]2CO[C@@](Cn3cncn3)(c3ccc(F)cc3F)C2)cc1]

### BACKGROUND OF THE INVENTION

U.S. Pat. No. 5,403,937 discloses a process for the preparation of key intermediate of Posaconazole, specifically ((3S,5R)-5-((1H-1,2,4-triazol-1-yl)methyl)-5-(2,4-difluorophenyl)tetrahydrofuran-3-yl)methyl-4-methylbenzene sulfonate. The process involves the usage of n-butyl lithium during the preparation of oxazolidinone lithium salt, which is extremely flammable. The said process requires column chromatographic purification at different stages to purify the intermediates which is tedious and lengthy process. The above said drawbacks make the process unviable on commercial scale.

In view of the above, there is an obvious need to find an efficient and industrially advantageous process for the synthesis of above said key intermediate of Posaconazole which overcomes the problems associated with the prior art such as prolonged reaction time, low yields and tedious purifications.

### BRIEF DESCRIPTION OF THE INVENTION

The first aspect of the present invention is to provide an improved process for the preparation of ((3S,5R)-5-((1H-1,2,4-triazol-1-yl)methyl)-5-(2,4-difluorophenyl)tetrahydrofuran-3-yl)methyl-4-methyl benzenesulfonate compound of formula-1.

The second aspect of the present invention is to provide, an improved-process, for the preparation of (R)-3-(4-(2,4-difluorophenyl)pent-4-enoyl)-4-phenyloxazolidin-2-one compound of formula-5.

The third aspect of the present invention is to provide a novel process for the preparation of ((3S,5R)-5-((1H-1,2,4-triazol-1-yl)methyl)-5-(2,4-difluorophenyl)tetrahydrofuran-3-yl)methyl-4-methylbenzenesulfonate compound of formula-1.

The fourth aspect of the present invention is to provide a process for the preparation of (2R,4R)-2-(2,4-difluorophenyl)-2-(iodomethyl)-4-(trityloxymethyl)tetrahydrofuran compound of formula-10 and also provides its novel crystalline form.

The fifth aspect of the present invention is to provide a process for the preparation of 1-(((2R,4R)-2-(2,4-difluorophenyl)-4-(trityloxymethyl)tetrahydrofuran-2-yl)methyl)-1H-1,2,4-triazole compound of formula-11 and also provides its novel crystalline form.

The sixth aspect of the present invention is to provide a process for the preparation of ((3R,5R)-5-((1H-1,2,4-triazol-1-yl)methyl)-5-(2,4-difluorophenyl)tetrahydrofuran-3-yl)methanol compound of formula-12.

The seventh aspect of the present invention is to provide novel intermediates which are useful in the preparation of triazole derivative compound of formula-1.

The eighth aspect of the present invention is to provide an improved process for the preparation of (R)-3-((3S,5R)-5-(2,4-difluorophenyl)-5-(iodomethyl)tetrahydrofuran-3-carbonyl)-4-phenyloxazolidin-2-one compound of formula-7.

The ninth aspect of the present invention is to provide a process for the purification of 1-(((2R,4R)-2-(2,4-difluorophenyl)-4-(trityloxymethyl)tetrahydrofuran-2-yl)methyl)-1H-1,2,4-triazole compound of formula-11.

The tenth aspect of the present invention is to provide an improved process for the preparation of amorphous 4-[4-[4-[4-[[(3R,5R)-5-(2,4-difluorophenyl)tetrahydro-5-(1H-1,2,4-triazol-1-ylmethyl)-3furanylimethoxy]phenyl]-1-piperazinyllphenyl]-2-[(1S,2S)-1-ethyl-2-hydroxypropyl]-2,4-dihydro-3H-1,2,4-triazol-3-one compound of formula-I, which comprising of:

a) Dissolving the compound of formula-I in a suitable solvent,

b) stirring the reaction mixture,

c) filtering the reaction mixture,

d) adding the filtrate to a suitable anti-solvent,

e) stirring the reaction mixture,

f) filtering the solid and then drying to get amorphous form of compound of formula-I.

### BRIEF DESCRIPTION OF THE DRAWINGS

FIG. 1: Illustrates the PXRD pattern of crystalline form-M of (2R,4R)-2-(2,4-difluorophenyl)-2-(iodomethyl)-4-(trityloxymethyl)tetrahydrofuran.

FIG. 2: Illustrates the DSC thermogram of crystalline form-M of (2R,4R)-2-(2,4-difluorophenyl)-2-(iodomethyl)-4-(trityloxymethyl)tetrahydrofuran.

FIG. 3: Illustrates the IR spectrum of crystalline form-M of (2R,4R)-2-(2,4-difluorophenyl)-2-(iodomethyl)-4-(trityloxymethyl)tetrahydrofuran.

FIG. 4: Illustrates the PXRD pattern of crystalline form-S of 1-(2R,4R)-2-(2,4-difluorophenyl)-4-(trityloxymethyl)tetrahydrofuran-2-yl)methyl)-1H-1,2,4-triazole.

FIG. 5: Illustrates the DSC thermogram of crystalline form-S of 1-(((2R,4R)-2-(2,4-difluorophenyl)-4-(trityloxymethyl)tetrahydrofuran-2-yl)methyl)-1H-1,2,4-triazole.

FIG. 6: Illustrates the IR spectrum of crystalline form-S of 1-(((2R,4R)-2-(2,4-difluorophenyl)-4-(trityloxymethyl)tetrahydrofuran-2-yl)methyl)-1H-1,2,4-triazole.

FIG. 7: Illustrates the PXRD pattern of amorphous compound of formula-I.

### DETAILED DESCRIPTION OF THE INVENTION

The term “suitable solvent” used in the present invention refers to “hydrocarbon solvents” such as n-hexane, n-pentane, n-heptane, cyclohexane, pet ether, benzene, toluene, xylene and the like; “ether solvents” such as dimethyl ether, diethyl ether, diisopropyl ether, methyl tert-butyl ether, 1,2-dimethoxy ethane, tetrahydrofuran, 1,4-dioxane and the like; “ester solvents” such as methyl acetate, ethyl acetate, n-propyl acetate, t-butyl acetate, iso-butyl acetate, isopropyl acetate, n-butyl acetate and the like; “polar-aprotic solvents such as dimethyl acetamide, dimethyl formamide, dimethyl sulfoxide, N-methyl pyrrolidone (NMP) and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcoholic solvents” such as methanol, ethanol, n-propanol, isopropanol, n-butanol, isobutanol, t-butanol and the like; “polar solvents” such as water or mixtures thereof.

As used herein the present invention, the term “anti-solvent” refers to a solvent which is used to precipitate the solid from a solution and the anti-solvents refers to solvents selected from “ether solvents” like tetrahydrofuran, diethyl ether, methyl tert-butyl ether and the like.

The term “suitable base” used in the present invention refers to inorganic bases selected from “alkali metal carbonates” such as sodium carbonate, potassium carbonate, lithium carbonate, cesium carbonate and the like; “alkali metal bicarbonates” such as sodium bicarbonate, potassium bicarbonate lithium bicarbonate and the like; “alkali metal hydroxides” such as sodium hydroxide, potassium hydroxide, lithium hydroxide and the like; “alkali metal alkoxides” such as sodium methoxide, sodium ethoxide, potassium methoxide, potassium ethoxide, sodium tert.butoxide, potassium tert.butoxide, lithium tert.butoxide and the like; “alkali metal hydrides” such as sodium hydride, potassium hydride, lithium hydride and the like; “alkali metal amides” such as sodium amide, potassium amide, lithium amide and the like; ammonia; and organic bases like methylamine, dimethylamine, diethylamine, diisopropyl amine, diisopropylethylamine, diisobutylamine, triethylamine, tributylamine, tert.butyl amine, pyridine, 4-dimethylaminopyridine (DMAP), N-methylmorpholine (NMM), 2,6-lutidine, lithium diisopropylamide (LDA), 1,8-diazabicyclo[5.4.0]undec-7-ene (DBU), 1,5-diazabicyclo[4.3.0]non-5-ene (DBN), 1,4-diazabicyclo[2.2.2]octane (DABCO) and the like; organosilicon bases such as lithium hexamethyl disilazide (LiHMDS), sodium hexamethyldisilazide (NaHMDS), potassium hexamethyldisilazide (KHMDS); n-butyl lithium or mixtures thereof.

The term suitable “reducing agent” used in the present invention refers to Ni, Raney Ni, Pd/C, Pt/C, PtO₂, Fe, Fe in acidic media like hydrochloric acid, acetic acid, NH₄Cl; Sn—HCl stannous chloride (SnCl₂), Zn in acidic media like hydrochloric acid, acetic acid, NH₄Cl, Zinc dust, DIBAL-H, lithium aluminium hydride, sodium borohydride, potassium borohydride, lithium borohydride, sodium aluminium hydride, diborane, hydrazine hydrate, sodium dithionate, sodium sulfide, ammonium sulfide, Na—Hg/H₂, borane-tetrahydrofuran, NaBH₃CN, sodium borohydride/BF₃-etherate, vitride, sodium borohydride/aluminium chloride or borane/aluminium chloride and sodium borohydride/iodine and 9-BBN.

The term suitable “deprotecting agent” used in the present invention refers to hydrochloric acid, aq. phosphoric acid, sulfuric acid, trifluoroacetic acid, methane sulfonic acid, acetyl chloride, Pd, Pd/C, Raney Ni, palladium acetate, platinum oxide, platinum black, Rh/C, Ru, Ir and the like in combination with hydrogen.

The term suitable “coupling agent” used in the present invention refers to N,N′-dicyclohexylcarbodiimide (DCC), N,N′-diisopropylcarbodiimide (DIC), 1-ethyl-3-(3-dimethylaminopropyl)carbodiimide hydrochloride (EDC.HCl), alkyl or aryl chloroformates such as ethyl chloroformate, benzylchloroformate, diphenyl phosphoroazidate (DPPA), thionyl chloride, oxalyl chloride, phosphorous oxychloride, phosphorous pentachloride, 4-methyl-2-oxopentanoyl chloride (i-BuCOCOCI), benzotriazol-1-yl-oxytripyrrolidino phosphonium hexafluorophosphate (PyBOP), methane sulfonyl chloride and the like optionally, in combination with 1-hydroxy-7-azatriazole (HOAt), 1-hydroxybenzotriazole (HOBt), 1 -hydroxy-1H-1,2,3-triazole-4-carboxylate (HOCt), O-(benzotriazol-1-yl)-N,N,N′,N′-tetramethyluronium tetrafluoroborate (TBTU), N-hydroxysuccinamide (HOSu), N-hydroxysulfo succinimide (Sulfo-NHS), 4-dimethylaminopyridine and the like.

The first aspect of the invention provides an improved process for the preparation of ((3S,5R)-5-((1H-1,2,4-triazol-1-yl)methyl)-5-(2,4-difluorophenyl)tetrahydrofuran-3-yl)methyl-4-methyl benzenesulfonate compound of formula-1, comprising of the following steps;
a) Reacting 4-(2,4-difluorophenyl)-4-oxobutanoic acid compound of formula-2 with methyl triphenyl phosphonium bromide in presence of a suitable base in a suitable solvent provides 4-(2,4-difluorophenyl)pent-4-enoic acid compound of formula-3,
b) reacting the compound of formula-3 with (R)-4-phenyloxazolidin-2-one compound of formula-4 in presence of suitable coupling agent in a suitable base in a suitable solvent provides (R)-3-(4-(2,4-difluorophenyl)pent-4-enoyl)-4-phenyloxazolidin-2-one compound of formula-5,
c) reacting the compound of formula-5 with 1,3,5-trioxane in presence of a suitable base and suitable catalyst in a suitable solvent provides (R)-3-(S)-4-(2,4-difluorophenyl)-2-(hydroxymethyl)pent-4-enoyl)-4-phenyloxazolidin-2-one compound of formula-6,
d) cyclizing the compound of formula-6 in-situ in presence of iodine and a suitable base in a suitable solvent provides (R)-3-(3S,5R)-5-(2,4-difluorophenyl)-5-(iodomethyl)tetrahydrofuran-3-carbonyl)-4-phenyloxazolidin-2-one compound of formula-7,
e) hydrolyzing the compound of formula-7 in presence of a suitable base and a suitable catalyst in a suitable solvent provides (3S,5R)-5-(2,4-difluorophenyl)-5-(iodomethyl)tetrahydrofuran-3-carboxylic acid compound of formula-8,
f) reducing the compound of formula-8 with a suitable reducing agent in a suitable solvent provides 3R,5R)-5-(2,4-difluorophenyl)-5-(iodomethyl)tetrahydrofuran-3-yl)methanol compound of formula-9,
g) reacting the compound of formula-9 with trityl chloride in presence of a suitable base in a suitable solvent provides (2R,4R)-2-(2,4-difluorophenyl)-2-(iodomethyl)-4-(trityloxymethyl) tetrahydrofuran compound of formula-10,
h) reacting the compound of formula-10 with 1H-1,2,4-triazole in the presence of a suitable base in a suitable solvent provides 1-(((2R,4R)-2-(2,4-difluorophenyl)-4-(trityloxymethyl) tetrahydrofuran-2-yl)methyl)-1H-1,2,4-triazole compound of formula-11, reacting the compound of formula-11 with a suitable deprotecting agent in a suitable solvent provides ((3R,5R)-5-((1H-1,2,4-triazol-1-yl)methyl)-5-(2,4-difluorophenyl)tetrahydrofuran-3-yl)methanol compound of formula-12,
j) reacting the compound of formula-12 with paratoluene sulfonyl chloride in presence of a base in a suitable solvent provides ((3S,5R)-5-((1H-1,2,4-triazol-1-yl)methyl)-5-(2,4-difluoro phenyl)tetrahydrofuran-3-yl)methyl 4-methylbenzenesulfonate compound of formula-1.

### Wherein,

in step-a) the suitable base is selected from organic or inorganic base; and the suitable solvent is selected from chloro solvents, ester solvents, ketone solvents, hydrocarbon solvents, polar aprotic solvents and alcohol solvents or mixtures thereof.
in step-b) the suitable coupling agent is selected from dicyclohexyl carbodiimide (DCC), diisopropylcarbodiimide (DIC), ethyl-(N″,N″-dimethylamino)inopylcarbodiimide hydrochloride (EDC), N, N-carbonyldiimidazole (CDI), DABAL-Me₃; and the suitable base is selected from organic or inorganic base; and the suitable solvent is selected from chloro solvents, ester solvents, ketone solvents, hydrocarbon solvents, polar aprotic solvents and alcohol solvents or mixtures thereof.
in step-c) the suitable base is selected from organic bases such as triethylamine, tributyl amine, pyridine, 4-dimethylaminopyridine, N-methyl morpholine and diisopropylethyl amine; and the suitable solvent is selected from alcohol solvents, chloro solvents, ketone solvents, ether solvents, ester solvents and mixture thereof; and the suitable catalyst is selected from Lewis acids such as TiCl₄.
in step-d) the suitable base, is selected from inorganic base such as alkali metal hydroxides, alkali metal alkoxides, alkali metal carbonates and alkali metal bicarbonates; and the suitable solvent is selected from chloro solvents, ether solvents, alcohol solvents, ester solvents, hydrocarbon solvents, ketone solvents and mixture thereof.
in step-e) the suitable catalyst is preferably hydrogen peroxide and the suitable base is selected from inorganic bases such as alkali metal hydroxides, alkali metal alkoxides, alkali metal carbonates, alkali metal bicarbonates and the suitable solvent is selected from ether solvents, alcohol solvents, ester solvents, ketone solvents, hydrocarbon solvents and mixtures thereof.
in step-f) the suitable reducing agent is selected from DIBAL-H, lithium aluminium hydride, sodium borohydride, lithium borohydride, NaBH₃CN, sodiumborohydride/BF₃-etherate, vitride, sodium borohydride/aluminium chloride or borane/aluminium chloride and sodium borohydride/iodine; and the suitable solvent is selected from ether solvents, chloro solvents, ester solvents, hydrocarbon solvents and ketone solvents, alcohol solvents or mixture thereof.
in step-g) the suitable base is selected from organic bases such as triethylamine, tributyl amine, pyridine, 4-dimethylaminopyridine, N-methyl morpholine and diisopropylethyl amine; and the suitable solvent is selected from alcohol solvents, chloro solvents, ketone solvents, ether solvents, ester solvents and mixture thereof.
in step-h) the suitable base is selected from inorganic base such as alkali metal hydroxides, alkali metal alkoxides, alkali metal carbonates, alkali metal bicarbonates or organic base such as triethylamine, tributyl amine, pyridine, 4-dimethylaminopyridine, N-methyl morpholine and diisopropylethyl amine; and the suitable solvent is selected from polar aprotic solvents, chloro solvents, alcoholic solvents ester solvents, hydrocarbon solvents and mixture thereof.
in step-i) the suitable deprotecting agent is selected from mineral acid such as sulfuric acid, hydrochloric acid, nitric acid, phosphoric acid, boric acid, hydrofluoric acid, hydrobromic acid, perchloric acid; the suitable solvent is selected from chloro solvents, ester solvents, ketone solvents, alcohol solvents, ether solvents, polar solvents such as water, polar aprotic solvents and mixture thereof.
in step j) the suitable base is selected from organic base as defined above and the suitable solvent is selected from chloro solvents, ester solvents, ketone solvents, alcoholic solvents, hydrocarbon solvents and mixture thereof.

The second aspect of the present invention provides an improved process for the preparation of (R)-3-(4-(2,4-difluorophenyl)pent-4-enoyl)-4-phenyloxazolidin-2-one compound of formula-5, comprising of, reacting 4-(2,4-difluorophenyl)pent-4-enoic acid compound of formula-3 with (R)-4-phenyloxazolidin-2-one compound of formula-4 in presence of a suitable coupling agent and a suitable base in a suitable solvent to provide (R)-3-(4-(2,4-difluorophenyl)pent-4-enoyl)-4-phenyloxazolidin-2-one compound of formula-5.

Wherein, the suitable coupling agent, the suitable base and the suitable solvents used are same as defined in step-b) of the first aspect of the present invention.

The third aspect of the present invention provides a novel process for the preparation of ((3S,5R)-5-((1H-1,2,4-triazol-1-yl)methyl)-5-(2,4-difluorophenyl)tetrahydrofuran-3-yl)methyl-4-methylbenzenesulfonate compound of formula-1, comprising of the following steps;
a) Reacting ((3R,5R)-5-(2,4-difluorophenyl)-5-(iodomethyl)tetrahydrofuran-3-yl)methanol compound of formula-9 with trityl chloride in presence of suitable base in a suitable solvent to provide (2R,4R)-2-(2,4-difluorophenyl)-2-(iodomethyl)-4-(trityloxymethyl)tetrahydrofuran compound of formula-10,
b) reacting the compound of formula-10 with 1H-1,2,4-triazole in the presence of a suitable base in a suitable solvent to provide 1-(((2R,4R)-2-(2,4-difluorophenyl)-4-(trityloxymethyl)tetrahydrofuran-2-yl)methyl)-1H-1,2,4-triazole compound of formula-11,
c) reacting the compound of formula-11 with a suitable deprotecting agent in a suitable solvent to provide ((3R,5R)-5-((1H-1,2,4-triazol-1-yl)methyl)-5-(2,4-difluorophenyl)tetrahydrofuran-3-yl)methanol compound of formula-12,
d) reacting the compound of formula-12 with p-toluenesulfonyl chloride in presence of base in a suitable solvent to provide ((3S,5R)-5-((1H-1,2,4-triazol-1-yl)methyl)-5-(2,4-difluorophenyl) tetrahydrofuran-3-yl)methyl 4-methylbenzenesulfonate compound of formula-1.

### Wherein,

in step-a) & step-d) the suitable base and the suitable solvents used are same as defined in step-g) and step-j) of the first aspect of the present invention.

in step-b) the suitable base and the solvents used are same as defined in step-h) of the first aspect of the present invention.

in step-c) the suitable deprotecting agent and the suitable solvents used are same as defined in step-i) of the first aspect of the present invention.

U.S. Pat. No. 5,403,937 disclosed a process for the preparation of ((3R,5R)-5-((1H-1,2,4-triazol-1-yl)methyl)-5-(2,4-difluorophenyl)tetrahydrofuran-3-yl)methanol compound of formula-12 by the reaction of ((3R,5R)-5-(2,4-difluorophenyl)-5-(iodomethyl)tetrahydrofuran-3-yl)methanol compound of formula-9 with 1H-1,2,4-triazole to produce compound of formula-12. It is observed that during the reaction the side chains of compound of formula-9, (one with hydroxy group and other with iodo group) undergoes internal cyclization, leading to the formation of a process impurity “(1R,5R)-5-(2,4-difluorophenyl)-3,6-dioxabicyclo[3.2.1]octane”,

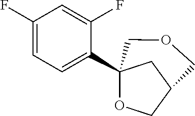 [SMILES: Fc1ccc([C@]23COC[C@H](CO2)C3)c(F)c1]

herein after designated as “cyclized impurity” to provide compound of formula-12 with low purity and yield.

Whereas the inventors of the present invention were able to overcome this problem and decreased the formation of the cyclized impurity to not detectable levels. This was achieved by the protection of hydroxy group of compound of formula-9 using hydroxy protecting group to provide (2R,4R)-2-(2,4-difluorophenyl)-2-(iodomethyl)-4-(trityloxymethyl)tetrahydrofuran compound of formula-10, which on subsequent reaction with 1H-1,2,4-triazole to provided compound of formula-11. Deprotection of the hydroxy protecting group provided (3R,5R)-5-((1H-1,2,4-triazol-1-yl)methyl)-5-(2,4-difluorophenyl)tetrahydrofuran-3-yl)methanol compound of formula-12 with high purity and yield. The present inventors observed that, by the protection of hydroxy group of compound of formula-9, they were able to prevent the internal cyclization of hydroxy group with the iodo group and thereby able to reduce the formation of cyclized impurity and which in lieu provided compound of formula-12 with high purity and greater yield. This impacts the subsequent reaction to provide compound of formula-1 with high purity and greater yield. Hence the present invention is advantageous over the prior art.

The fourth aspect of the present invention provides a process for the preparation of (2R,4R)-2-(2,4-difluorophenyl)-2-(iodomethyl)-4-(trityloxymethyl)tetrahydrofuran compound of formula-10, comprising of, reacting ((3R,5R)-5-(2,4-difluorophenyl)-5-(iodomethyl)tetrahydrofuran-3-yl)methanol compound of formula-9 with trityl chloride in presence of suitable base in a suitable solvent provides compound of formula-10.

Wherein, the suitable base and the suitable solvents used are same as defined in step-g) of the first aspect of the present invention.

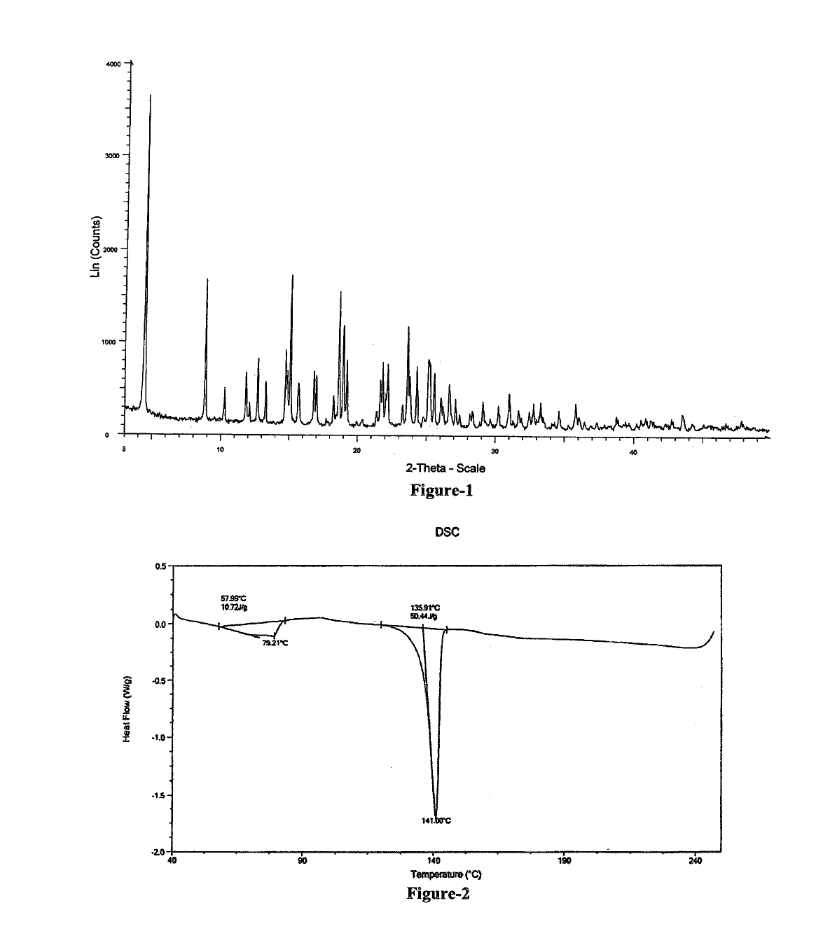

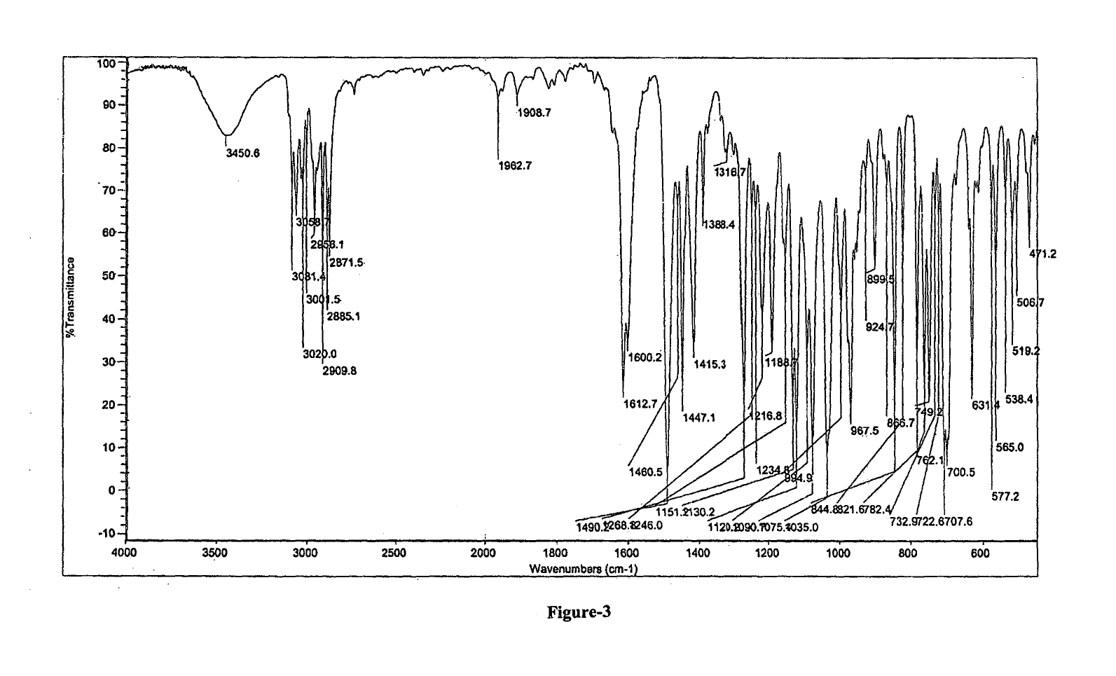

Further, the present invention provides novel crystalline form herein designated as form-M of (2R,4R)-2-(2,4-difluorophenyl)-2-(iodomethyl)-4-(trityloxymethyl)tetrahydrofuran, which is characterized by:
a) Its powder X-ray diffractogram having peaks at 4.3, 8.8, 10.1, 11.8, 12.1, 12.6, 13.2, 14.7, 15.0, 15.6, 16.7, 16.9, 18.2, 18.6, 18.9, 19.2, 19.4, 21.7, 22.1, 23.2, 23.5, 24.2, 25.1, 25.5, 26.0, 26.6, 27.1, 29.0, 30.2, 30.9, 31.6, 32.4, 32.7, 33.2, 34.6, 35.8 and 43.5±0.2 degrees of two-theta as illustrated in FIG. 1;
b) its DSC thermogram showing sharp endotherm at 141.00° C. as illustrated in FIG. 2.
c) its IR spectrum having absorption bands as illustrated in FIG. 3.

The fifth aspect of the present invention provides a process for the preparation of 1-(((2R,4R)-2-(2,4-difluorophenyl)-4-(trityloxymethyl)tetrahydrofuran-2-yl)methyl)-1H-1,2,4-triazole compound of formula-11, comprising of reacting (2R,4R)-2-(2,4-difluorophenyl)-2-(iodomethyl)-4-(trityloxymethyl)tetrahydrofuran compound formula-10 with 1H-1,2,4-triazole in presence of a suitable base in a suitable solvent provides compound of formula-11.

Wherein, the suitable base and the solvents used are same as defined in step-h) of the first aspect of the present invention.

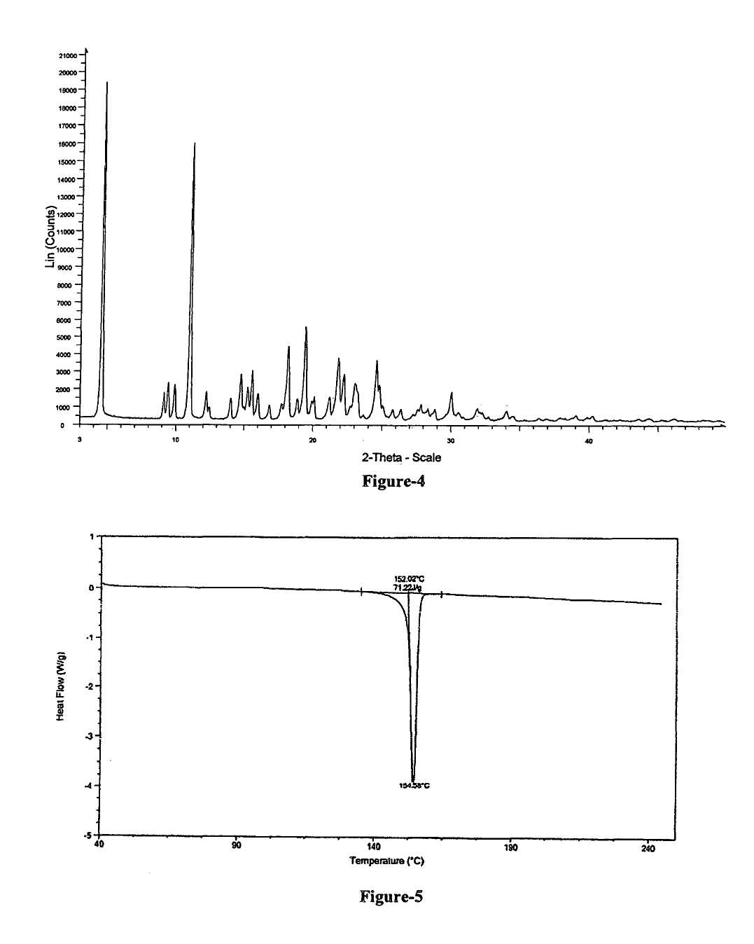

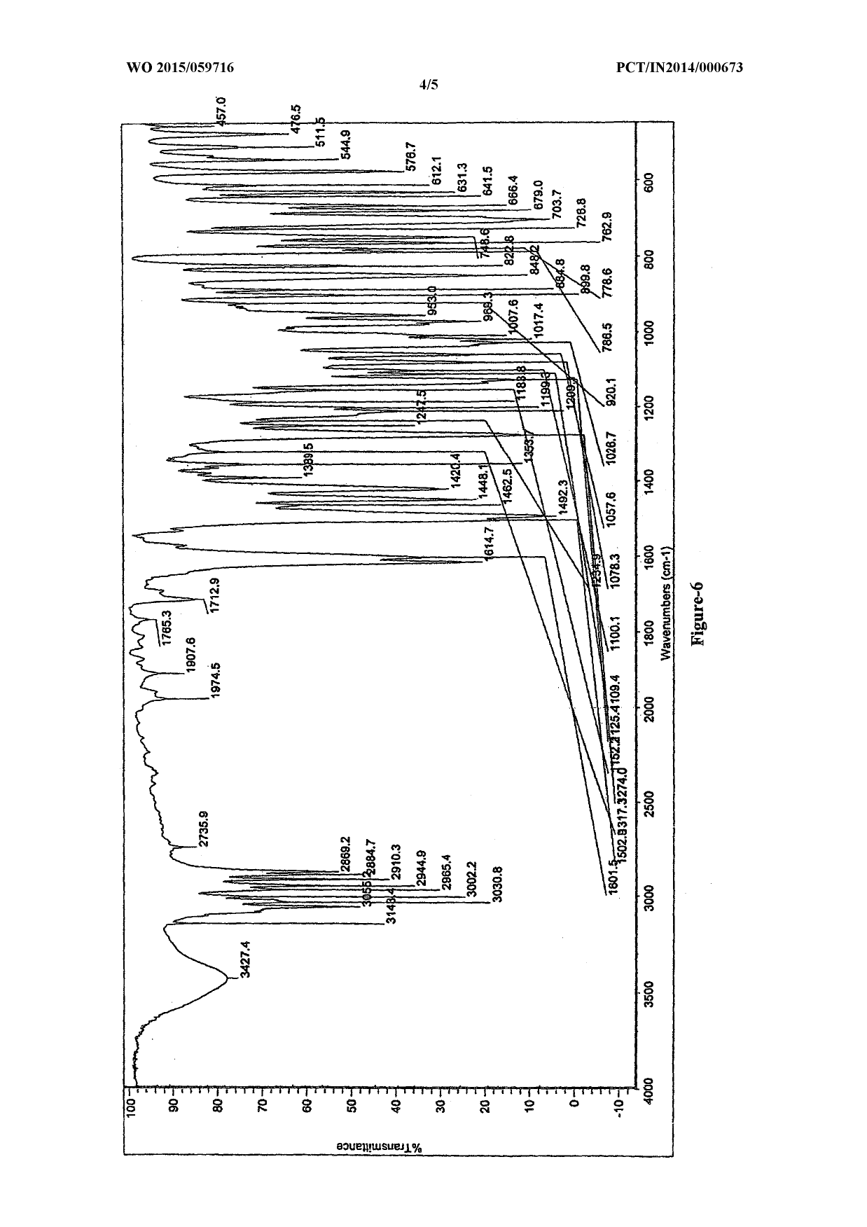

Further; the present invention provides novel crystalline form herein designated as form-S of 1-(((2R,4R)-2-(2,4-difluorophenyl)-4-(trityloxymethyl)tetrahydrofuran-2-yl)methyl)-1H-1,2,4-triazole, which is characterized by:
a) Its powder X-ray diffractogram having peaks at 4.5, 9.0, 9.4, 9.8, 10.9, 12.1, 13.9, 14.7, 15.2, 15.5, 15.9, 18.8, 19.4, 20.0, 21.1, 21.8, 22.2, 23.0, 24.5, 24.8, 27.8 and 30.0±0.2 degrees of two-theta as illustrated in FIG. 4;
b) its DSC thermogram showing sharp endotherm at 154.58° C. as illustrated in FIG. 5.
c) its IR spectrum having absorption bands as illustrated in FIG. 6.

The sixth aspect of the present invention provides a process for the preparation of ((3R,5R)-5-((1H-1,2,4-triazol-1-yl)methyl)-5-(2,4-difluorophenyl)tetrahydrofuran-3-yl)methanol compound of formula-12, comprising of reacting 1-(((2R,4R)-2-(2,4-difluorophenyl)-4-(trityloxymethyl)tetrahydrofuran-2-yl)methyl)-1H-1,2,4-triazole compound of formula-11 with a suitable deprotecting agent in a suitable solvent provides compound of formula-12.

Wherein, the suitable deprotecting agent and the suitable solvent used are same-as defined in step-i) of the first aspect of the present invention.

The preferred embodiment of the present invention provides a process for the preparation of ((3R,5R)-5-((1H-1,2,4-triazol-1-yl)methyl)-5-(2,4-difluorophenyl)tetrahydrofuran-3-yl)methanol compound of formula-12, comprising of reacting 1-(((2R,4R)-2-(2,4-difluorophenyl)-4-(trityloxymethyl)tetrahydrofuran-2-yl)methyl)-1H-1,2,4-triazole compound of formula-11 with sulfuric acid in acetone provides compound of formula-12.

In another embodiment of the present invention provides a process for the preparation of ((3R,5R)-5-((1H-1,2,4-triazol-1-yl)methyl)-5-(2,4-difluorophenyl)tetrahydrofuran-3-yl)methanol compound of formula-12, comprising of reacting 1-(((2R,4R)-2-(2,4-difluorophenyl)-4-(trityloxymethyl)tetrahydrofuran-2-yl)methyl)-1H-1,2,4-triazole compound of formula-11 with hydrochloric acid in methanol provides compound of formula-12.

The seventh aspect of the present invention provides novel intermediates which are useful in the preparation of triazole derivative compound of formula-1.

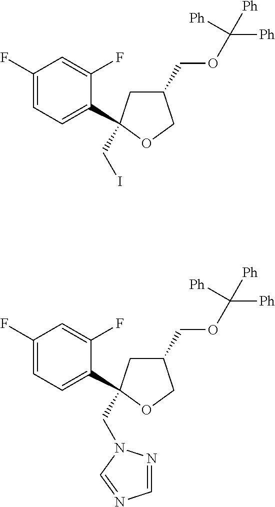 [SMILES: Fc1ccc([C@@]2(CI)C[C@H](COC(c3ccccc3)(c3ccccc3)c3ccccc3)CO2)c(F)c1 ; Fc1ccc([C@@]2(Cn3cncn3)C[C@H](COC(c3ccccc3)(c3ccccc3)c3ccccc3)CO2)c(F)c1]

The novel compounds i.e., (2R,4R)-2-(2,4-difluorophenyl)-2-(iodomethyl)-4-(trityloxymethyl)tetrahydrofuran compound of formula-10 and 1-(((2R,4R)-2-(2,4-difluoro phenyl)-4-(trityloxymethyl)tetrahydrofuran-2-yl)methyl)-1H-1,2,4-triazole compound of formula-11 and its crystalline polymorphs are useful in the preparation of compound of formula-1, further the compound of formula-1 is useful in the preparation of triazole anti-fungal drug Posaconazole.

The eighth aspect of the present invention is to provide an improved process for the preparation of (R)-3-((3S,5R)-5-(2,4-difluorophenyl)-5-(iodomethyl)tetrahydrofuran-3-carbonyl)-4-phenyloxazolidin-2-one compound of formula-7, comprising of:
a) Reacting the compound of formula-5 with 1,3,5-trioxane in presence of a suitable base and suitable catalyst in a suitable solvent provides (R)-3-((S)-4-(2,4-difluorophenyl)-2-(hydroxymethyl)pent-4-enoyl)-4-phenyloxazolidin-2-one compound of formula-6,
b) cyclizing the compound of formula-6 in-situ in presence of iodine in a suitable solvent provides (R)-3-((3S,5R)-5-(2,4-difluorophenyl)-5-(iodomethyl)tetrahydrofuran-3-carbonyl)-4-phenyloxazolidin-2-one compound of formula-7.

The preferred embodiment of the present invention is to provide an improved process for the preparation of (R)-3-((3S,5R)-5-(2,4-difluorophenyl)-5-(iodomethyl)tetrahydrofuran-3-carbonyl)-4-phenyloxazolidin-2-one compound of formula-7, comprising of:
a) Reacting the compound of formula-5 with 1,3,5-trioxane in presence of diisopropyl ethyl amine and titanium tetra chloride in dichloromethane provides (R)-3-((S)-4-(2,4-difluorophenyl)-2-(hydroxymethyl)pent-4-enoyl)-4-phenyloxazolidin-2-one compound of formula-6,
b) cyclizing the compound of formula-6 in-situ in presence of iodine in dichloromethane provides (R)-3-((3S,5R)-5-(2,4-difluorophenyl)-5-(iodomethyl)tetrahydrofuran-3-carbonyl)-4-phenyloxazolidin-2-one compound of formula-7.

The ninth aspect of the present invention is to provide a process for the purification of 1-(((2R,4R)-2-(2,4±-difluorophenyl)-4-(trityloxymethyl)tetrahydrofuran-2-yl)methyl)-1H-1,2,4-triazole compound of formula-11, Comprising of:
a) Dissolving 1-(((2R,4R)-2-(2,4-difluorophenyl)-4-(trityloxymethyl)tetrahydrofuran-2-yl)methyl)-1H-1,2,4-triazole compound of formula-11 in a suitable solvent,
b) heating the reaction mixture,
c) stirring the reaction mixture,
d) cooling the reaction mixture,
e) stirring the reaction mixture,
f) filtering the solid and drying to get pure 1-(((2R,4R)-2-(2,4-difluorophenyl)-4-(trityloxymethyl)tetrahydrofuran-2-yl)methyl)-1H-1,2,4-triazole compound of formula-11.

The preferred embodiment of the present invention provides a process for the purification of 1-(2R,4R)-2-(2,4-difluorophenyl)-4-(trityloxymethyl)tetrahydrofuran-2-yl)methyl)-1H-1,2,4-triazole compound of formula-11, comprising of:
a) Dissolving 1-(((2R,4R)-2-(2,4-difluorophenyl)-4-(trityloxymethyl)tetrahydrofuran-2-yl)methyl)-1H-1,2,4-triazole compound of formula-11 in acetone,
b) heating the reaction mixture,
c) stirring the reaction mixture,
d) cooling the reaction mixture,
e) stirring the reaction mixture,
f) filtering the solid and drying to get pure 1-(((2R,4R)-2-(2,4-difluorophenyl)-4-(trityloxymethyl)tetrahydrofuran-2-yl)methyl)-1H-1,2,4-triazole compound of formula-11.

The tenth aspect of the present invention provides an improved process for the preparation of amorphous 4-[4-[4-[4-[[(3R,5R)-5-(2,4-difluorophenyl)tetrahydro-5-(1H-1,2,4-triazol-1-ylmethyl)-3furanyl]methoxy]phenyl]-1-piperazinyl]phenyl]-2-[(1S,2S)-1-ethyl-2-hydroxypropyl]-2,4-dihydro-3H-1,2,4-triazol-3-one compound of formula-I, which comprising of:
a) Dissolving the compound of formula-I in a suitable solvent,
b) stirring the reaction mixture,
c) filtering the reaction mixture,
d) adding the filtrate to a suitable anti-solvent,
e) stirring the reaction mixture,
f) filtering the solid and then drying to get amorphous form of compound of formula-I.

Wherein, the suitable solvent used in step-a) is selected from chloro solvents, ketone solvents, ester solvents, alcoholic solvents and the mixtures thereof and the suitable anti-solvent used in step-d) is selected from ether solvents.

A preferred embodiment of the present invention provides an improved process for the preparation of amorphous 4-[4-[4-[4-[[(3R,5R)-5-(2,4-difluorophenyl)tetrahydro-5-(1H-1,2,4-triazol-1-ylmethyl)-3furanyl]methoxy]phenyl]-1-piperazinyl]phenyl]-2-[(1S,2S)-1-ethyl-2-hydroxypropyl]-2,4-dihydro-3H-1,2,4-triazol-3-one compound of formula-I, which comprising of:

a) Dissolving the compound of formula-I in dichloromethane,

b) stirring the reaction mixture,

c) filtering the reaction mixture,

d) adding the filtrate to methyl tertiary butyl ether (MTBE),

e) stirring the reaction mixture,

f) filtering the solid and then drying to get amorphous form of compound of formula-I.

The conversion of the compound of formula-1 to posaconazole can be carried out to any of the methods known in the art.

### P-XRD Method of Analysis:

PXRD analysis of compound of formula-10 and compound of formula-11 produced by the present invention was carried out using BRUKER/AXS X-Ray diffractometer using Cu Kα radiation of wavelength 1.5406 A° and continuous scan speed of 0.03°/min.

Differential scanning-catorimenic (DSC) analysis was performed with Q10 V9:6 Build 290 calorimeter. Samples of about 2 to 3 milligrams held in a closed pan were analyzed at a heating rate of 10° C. per minute.

### HPLC Method of Analysis:

Related substances of the Posaconazole intermediates were analyzed by HPLC using the following conditions:

### (2R,4R)-2-(2,4-difluorophenyl)-2-(iodomethyl)-4-(trityloxymethyl)tetrahydrofuran (Formula-10) and 1-(((2R,4R)-2-(2,4-difluorophenyl)-4-(trityloxymethyl)tetrahydrofuran-2-yl)methyl)-111-1,2,4-triazole (Formula-11)

Apparatus: A liquid chromatographic system is to be equipped with variable wavelength UV-detector; Column: Xterra RPB, 150×4.6 mm, 3.5 or equivalent; Flow rate: 1.2 ml/min; Wavelength: 210 nm; Column Temperature: 30° C.; Injection volume: 10 μL; Run time: 38 min; Diluent: Acetonitrile; Needle wash: Diluent; Elution: Gradient; Mobile phase-A: Buffer; sample concentration: 1.0 mg/ml; Mobile phase-B: Acetonitrile: Water (90:10%) v/v; Buffer: 2.0 ml of ortho phosphoric acid in 1000 ml of Milli-Q-water and filter through 0.22 μm filter paper.

### ((3S,5R)-5-(1H-1,2,4-triazol-1-yl)methyl)-5-(2,4-difluorophenyl)tetrahydrofuran-3-yl)methyl-4-methylbenzenesulfonate (Formula-1)

Apparatus: A liquid chromatographic system is to be equipped with variable wavelength UV-detector; Column: Zorbax SB C18, 250×4.6 mm, 5 μm or equivalent; Flow rate: 1.2 ml/min; Wavelength: 210 nm; Column Temperature: 30° C.; Injection volume: 10 μL; Run time: 48 min; Diluent: Acetonitrile; Needle wash: Diluent; Elution: Gradient; Mobile phase-A: Buffer; Mobile phase-B: Acetonitrile: Water (90:10%) v/v; Buffer: 0.2 ml of ortho phosphoric acid in 1000 ml of Milli-Q-water and filter through 0.22 μm Nylon membrane filter paper.

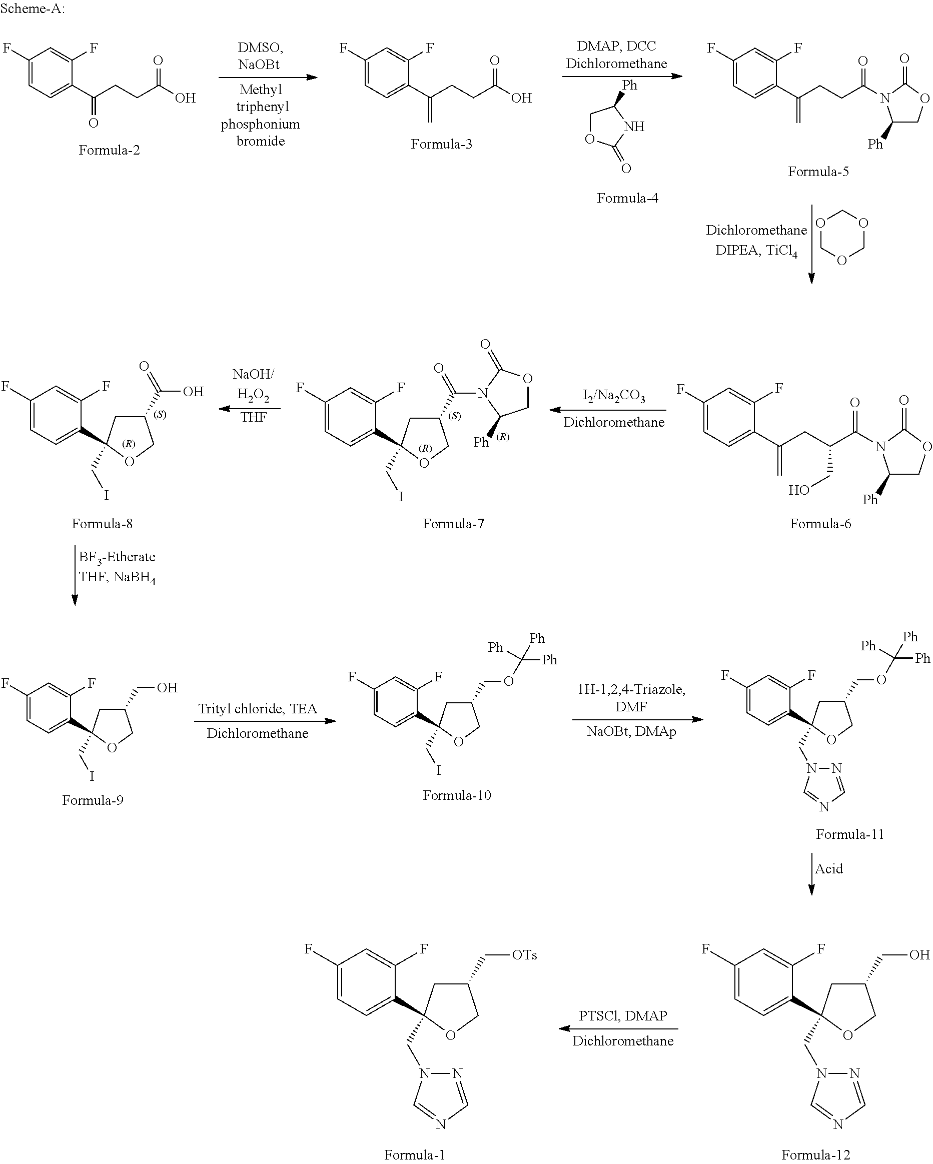

The best mode of carrying out the present invention is illustrated by the below mentioned examples. These examples are provides as illustration only and hence should not be construed as limitation of the scope of the invention.

### EXAMPLES

### Example-1

### Preparation of 4-(2,4-difluorophenyl)pent-4-enoic acid (Formula-3)

Methyl triphenylphoshonium bromide (250 gm) and dimethylsulfoxide (500 ml) was added to 4-(2,4-difluorophenyl)-4-oxobutanoic acid (100 gm) at 25-30° C. Cooled the reaction mixture to 10-15° C. Sodium tertiary butoxide (112 gm) was slowly added to the reaction mixture at 10-15° C. Raised the temperature of the reaction mixture to 25-30° C. and stirred for 2 hours at the same temperature. After completion of the reaction, cooled the reaction mixture to 10-15° C. and water was added to the mixture the reaction mixture at the same temperature. Further cooled the reaction mixture to 0-5° C. and stirred for 1 hour at the same temperature. Filtered the reaction mixture and washed with water at 0-5° C. Heated the obtained filtrate to 25-30° C. and washed with dichloromethane at the same temperature. The reaction mixture was cooled to 0-5° C. Adjusted the pH of the reaction mixture using aqueous hydrochloric acid solution at 0-5° C. and stirred for 1 hour at the same temperature. Filtered the precipitated solid, washed with water and dried to get the title compound. Yield: 92 gm.

### Example-2

### Preparation of 4-(2,4-difluorophenyl)pent-4-enoic acid (Formula-3)

Methyl triphenylphoshonium iodide (220 gm) and dimethylsulfoxide (500 ml) was added to 4-(2,4-difluorophenyl)-4-oxobutanoic acid (100 gm) at 25-30° C. Cooled the reaction mixture to 10-15° C. Sodium tertiary butoxide (112 gm) was slowly added to the reaction mixture at 10-15° C. Raised the temperature of the reaction mixture to 25-30° C. and stirred for 2 hours at the same temperature. After completion of the reaction, cooled the reaction mixture to 10-15° C. Water was slowly added to the reaction mixture at 10-15° C. Further cooled the reaction mixture to 0-5° C. and stirred for 1 hour at the same temperature. Filtered the reaction mixture and washed with water. Heated the obtained filtrate to 25-30° C. and washed with dichloromethane. The aqueous layer and cooled to 0-5° C. Adjusted the pH of the aqueous layer to 2-3 using dilute hydrochloric acid. Stirred the reaction mixture to 1 hour at 0-5° C. Filtered the precipitated solid, washed with water. To the obtained wet compound, water was added at 25-30° C. and stirred for 45 minutes at the same temperature. Filtered the solid, washed with water and dried to get the title compound. Yield: 95 gm.

### Example-3

### Preparation of (R)-3-(4-(2,4-difluorophenyl)pent-4-enoyl)-4-phenyloxazolidin-2-one (Formula-5)

Dichloromethane (150 ml) was added to 4-(2,4-difluorophenyl)pent-4-enoic acid (50 gm) at 25-30° C. Dimethyl aminopyridine (3.0 gm) and (R)-4-phenyloxazolidin-2-one (38.5 gm) were added to the reaction mixture at 25-30° C. Cooled the reaction mixture to 10-15° C. Dicyclohexylcarbodiimide (53.6 gm) was slowly added to the reaction mixture at 10-15° C. and stirred for 5 hours at the same temperature. After completion of the reaction, filtered the reaction mixture and washed with dichloromethane. To the obtained filtrate dilute hydrochloric acid was added at 25-30° C. and stirred for 15 minutes at the same temperature. Both the organic and aqueous layers were separated. Organic layer was washed with aqueous sodium bicarbonate solution and followed by washed with sodium chloride solution. Distilled off the solvent completely from the organic layer and cooled to 25-30° C. Isopropanol (100 ml) was added to the obtained residue at 25-30° C. and stirred for 15 minutes at the same temperature. Cooled the reaction mixture to 0-5° C. and stirred for 2 hours at the same temperature. Filtered the precipitated solid, washed with isopropanol and dried to get the title compound. Yield: 72 gm.

### Example-4

### Preparation of (R)-34(S)-4-(2,4-difluorophenyl)-2-(hydroxymethyl)pent-4-enoyl)-4-phenyloxazolidin-2-one (Formula-6)

Dichloromethane (1000 ml) was added to (R)-3-(4-(2,4-difluorophenyl)pent-4-enoyl)-4-phenyloxazolidin-2-one (100 gm) at 25-30° C. under nitrogen atmosphere. Cooled the reaction mixture to −10-15° C. Titanium tetrachloride solution in dichloromethane (50 ml) was slowly added to the reaction mixture at −10-15° C. Diisopropyl ethylamine (45.61 gm) was added to the reaction mixture at −10-15° C. and stirred for 1 hour at the same temperature. 1,3,5-Trioxane solution in 150 ml of dichloromethane was added to the reaction mixture at −10-15° C. Titanium tetrachloride solution (40 ml) and dichloromethane (50 ml) were slowly added to the reaction mixture at −10-15° C. Raised the temperature of the reaction mixture to 0-5° C. and stirred for 2 hours at the same temperature. After completion of the reaction, quenched the reaction mixture using 10% ammonium chloride solution. Both the organic and aqueous layers were separated and organic layer was washed with water, followed by 10% sodium chloride solution. The organic layer containing (R)-3-((S)-4-(2,4-difluorophenyl)-2-(hydroxymethyl)pent-4-enoyl)-4-phenyloxazolidin-2-one is used in the next step without isolation.

### Example-5

### (R)-34(3S,5R)-5-(2,4-difluorophenyl)-5-(iodomethyl)tetrahydrofuran-3-carbonyl)-4-phenyloxazolidin-2-one (Formula-7)

Sodium carbonate (59.4 gm) was added to the organic layer obtained in the example-4 at 25-30° C. Iodine (177.8 gm) was added to the reaction mixture at 25-30° C. and stirred for 2 hours at the same temperature. After completion of the reaction, quenched the reaction mixture using 30% sodium thiosulphate solution at 25-30° C. Both the organic and aqueous layers were separated and distilled off the solvent from the organic layer at 40-45° C. Methyl tertiary butyl ether (1:000 ml) was added to the obtained compound at 40-45° C. and stirred for 15 minutes at the same temperature. The reaction mixture is washed with 30% sodium thiosulphate solution, followed by 10% sodium chloride solution. Distilled off the solvent from the reaction mixture at 40-45° C. Methyl tertiary butyl ether (500 ml) was added to the obtained crude compound. Cooled the reaction mixture to 0-5° C. and stirred for 3 hours at the same temperature. Filtered the precipitated solid, washed with methyl tertiary butyl ether and dried to get the title compound.

Yield: 75 gms. Purity by HPLC: 98%.

### Example-6

### Preparation of (3S,5R)-5-(2,4-difluorophenyl)-5-(iodomethyl)tetrahydrofuran-3-carboxylic acid (Formula-8)

Hydrogen peroxide (23.2 ml) was added to a pre-cooled solution of sodium hydroxide (13.6 gm) and water (100 ml) at 0-5° C. A mixture of (R)-3-(3S,5R)-5-(2,4-difluorophenyl)-5-(iodomethyl)tetrahydrofuran-3-carbonyl)-4-phenyloxazolidin-2-one (100 gm) and tetrahydrofuran (250 ml) was added to the reaction mixture at 0-5° C. and stirred for 3 hour at the same temperature. After completion of the reaction, quenched the reaction mixture using 10% sodium sulphite solution at 0-5° C. Water was added to the reaction mixture at 0-5° C. Raised the temperature of the reaction mixture to 25-30° C. Both the organic and aqueous layers were separated and aqueous layer was washed with dichloromethane. Adjusted the pH of the aqueous layer to 7.5 using 20% hydrochloric acid solution. Adjusted the pH of the aqueous layer from 4.0-5.0 using 20% hydrochloric acid solution and stirred for 2 hours at 25-30° C. Cooled the aqueous layer to 0-5° C. and stirred for 2 hours at the same temperature. Filtered the precipitated solid, washed with water and dried to get the title compound.

Yield: 82 gm; Purity by HPLC: 97.5%.

### Example-7

### Preparation of ((3R,5R)-5-(2,4-difluorophenyl)-5-(iodomethyl)tetrahydrofuran-3-yl)methanol (Formula-9)

A mixture of (3S,5R)-5-(2,4-difluorophenyl)-5-(iodomethyl)tetrahydrofuran-3-carboxylic acid (250 gm) and tetrahydrofuran (500 ml) were slowly added to a pre-cooled solution of tetrahydrofuran (500 ml) and sodium borohydride (52 gm) at 0-5° C. 48% BF₃-etherate solution to the reaction mixture at 0-5° C. under nitrogen atmosphere. Raised the temperature of the reaction mixture to 25-30° C. and stirred for 2 hours at the same temperature. After completion of the reaction, chilled water was-added to the reaction mixture 25-30° C. The reaction mixture was extracted with dichloromethane. The organic layer was washed with 5% hydrogen peroxide solution followed by washed with 5% sodium sulphite solution, followed by sodium chloride solution. Distilled off the solvent completely from the organic layer to get the title compound.

Yield: 225 gm; Purity by HPLC: 98.2%.

### Example-8

### Preparation of (2JR,4R)-2-(2,4-difluorophenyl)-2-(iodomethyl)-4-(trityloxymethyl)tetrahydrofuran (Formula-10)

Trityl chloride (256 gm) was added to a mixture of dichloromethane (1250 ml) and ((3R,5R)-5-(2,4-difluorophenyl)-5-(iodomethyl)tetrahydrofuran-3-yl)methanol (250 gm) at 25-30° C. and stirred the reaction mixture for 15 minutes at the same temperature. Triethyl amine (107 gm) was slowly added to the reaction mixture at 25-30° C. and stirred for 2 hours at the same temperature. After completion of the reaction, water was added to the reaction mixture at 25-30° C. and stirred for 15 minutes at the same temperature. Both the organic and aqueous layers were separated and organic layer is washed with sodium chloride solution. Distilled off the solvent completely from the organic layer and co distilled with methanol. Methanol (1000 ml) was added to the obtained crude compound at 25-30° C. and stirred for 3 hours at the same temperature. Cooled the reaction mixture to 5-10° C. and stirred for 45 minutes at the same temperature. Filtered the precipitated solid, washed with methanol and dried to get the title compound. Yield: 355 gm; M.R: 136-138° C.; Purity by HPLC: 99.93%.

The P-XRD of the obtained compound is shown in FIG. 1.

### Example-9

### 1-(((2R,4R)-2-(2,4-difluorophenyl)-4-(trityloxymethyl)tetrahydrofuran-2-yl)methyl)-1H-1,2,4-triazole (Formula-11)

Sodium tertiary butoxide (100 gm) was added to a mixture of dimethyl formamide (1250 ml), 1H-1,2,4-triazole (72 gm) and dimethylaminopyridine (5 gm) at 25-30° C. (2R,4R)-2-(2,4-difluorophenyl)-2-(iodomethyl)-4-(trityloxymethyl)tetrahydrofuran (250 gm) were added to the above reaction mixture at 25-30° C. Heated the reaction mixture to 110-115° C. and stirred for 12 hours at the same temperature. After completion of the reaction, cooled the reaction mixture to 25-30° C. Water was slowly added to the reaction mixture at 25-30° C. and stirred for 45 minutes at the same temperature. Filtered the precipitated solid, washed with water and dried to get the title compound. Yield: 212 gm; M.R: 150-152° C.; Purity by HPLC: 99.97%.

The P-XRD-of the obtained compound is shown in FIG. 4.

### Example-10

### Preparation of ((3R,5R)-5-((1H-1,2,4-triazol-1-yl)methyl)-5-(2,4-difluorophenyl)tetrahydrofuran-3-yl)methanol (Formula-12)

Dilute sulfuric acid {prepared by adding water (100 ml) to sulfuric acid (228 gm)} was slowly added to a pre-cooled solution of acetone (750 ml) and 1-(2R,4R)-2-(2,4-difluorophenyl)-4-(trityloxymethyl)tetrahydrofuran-2-yl)methyl)-1H-1,2,4-triazole (250 gm) at 10-15° C. Raised the temperature of the reaction mixture to 25-30° C. and stirred for 3 hours at the same temperature. Stirred the reaction mixture for further 45 minutes at 25-30° C. Water (1500 ml) was added added to the reaction mixture and filtered the reaction mixture. Filtrate was washed with cyclohexane at 25-30° C. Aqueous layer was cooled to 10-15° C. and adjusted the pH to 9 using aqueous sodium hydroxide. Extracted the aqueous layer with ethyl acetate. Organic layer was washed with 10% sodium chloride solution. Distilled off the solvent completely from the organic layer to get the title compound. Yield: 137 gm.

### Example-11

### Preparation of ((3R,5R)-54(1H-1,2,4-triazol-1-yl)methyl)-5-(2,4-difluorophenyl)tetrahydrofuran-3-yl)methanol (Formula-12)

Dilute hydrochloric acid (80 ml) was added to a pre-cooled solution of acetone (750 ml) and 1-(2R,4R)-2-(2,4-difluorophenyl)-4-(trityloxymethyl)tetrahydrofuran-2-yl)methyl)-1H-1,2,4-triazole (250 gm) at 10-15° C. Raised the temperature of the reaction mixture to 25-30° C. and stirred for 2 hours at the same temperature. Water was slowly added to the reaction mixture at 25-30° C. and stirred for 45 minutes at the same temperature. Filtered the reaction mixture and washed with water. The obtained filtrate was washed with cyclohexane. Aqueous layer was cooled to 10-15° C. and adjusted the pH to 9 using aqueous sodium hydroxide. Extracted the aqueous layer with ethyl acetate. Organic layer was washed with 10% sodium chloride solution. Distilled off the solvent completely from the organic layer to get the title compound.

Yield: 134 gm.

Example-12

### Preparation of ((3S,5R)-5-(1H-1,2,4-triazol-1-yl)methyl)-5-(2,4-difluorophenyl)tetrahydrofuran-3-yl)methyl 4-methylbenzenesulfonate (Formula-1)

Dimethyl aminopyridine (114 gms), followed by para toluene sulfonyl chloride (161 gms) were slowly added to a pre-cooled solution of dichloromethane (1250 ml) and ((3R,5R)-5-((1H-1,2,4-triazol-1-yl)methyl)-5-(2,4-difluorophenyl) tetrahydrofuran-3-yl)methanol (250 gms) at 0-5° C. Raised the temperature of the reaction mixture to 25-30° C. and stirred for 8 hours at the same temperature. Water was added to the reaction mixture at 25-30° C. and stirred for 15 minutes at the same temperature. Both the organic and aqueous layers were separated and washed the organic layer with 10% aqueous hydrochloric acid solution, followed by 10% aqueous sodium carbonate solution. Organic layer is washed with water. Distilled off the solvent from the organic layer and co-distilled with petroleum ether. Ethanol (100 ml) were added to the obtained solid at 25-30° C. and heated to 60-65° C. and then cooled to 25-30° C. and stirred for 8 hrs at the same temperature. Further, cooled to 0-5 and filtered the precipitated solid, washed with ethanol and then dried to get the title compound. Yield: 215 gms. Purity by HPLC: 99.98%.

### Example-13

### Preparation of ((3S,5R)-5-(1H-1,2,4-triazol-1-yl)methyl)-5-(2,4-difluorophenyl)tetrahydrofuran-3-yl)methyl 4-methylbenzenesulfonate (Formula-1)

Para toluene sulfonyl chloride (161 gm) was added to a solution of acetone (750 ml) and ((3R,5R)-5-((1H-1,2,4-triazol-1-yl)methyl)-5-(2,4-difluorophenyl)tetrahydrofuran-3-yl)methanol (250 gm) at 15-20° C. Sodium hydroxide solution was slowly added to the reaction mixture at 25-30° C. and stirred for 4 hours at the same temperature. Water was added to the reaction mixture at 25-30° C. and stirred for 2 hours at the same temperature. Filtered the precipitated solid and washed with water and dried to get the title compound. Yield: 169 gm; Purity by HPLC: 99.95%.

### Example-14

### One-Pot Process for the Preparation of (R)-3-((3S,5R)-5-(2,4-difluorophenyl)-5-(iodomethyl)tetrahydrofuran-3-carbonyl)-4-phenyloxazolidin-2-one (Formula-7):

Titanium tetrachloride solution [prepared by adding titanium tetrachloride (33.8 ml) and dichloromethane (50 ml)] was added to a pre-cooled mixture of dichloromethane (1000 ml) and (R)-3-(4-(2,4-difluorophenyl)pent-4-enoyl)-4-phenyloxazolidin-2-one (100 gms) compound of formula-5 under nitrogen atmosphere at −20 to −15° C. Diisopropyl ethyl amine (45.61 gms) was slowly added to the reaction mixture at −20 to −15° C. and stirred for 1 hour at the same temperature. 1,3,5-trioxane solution [prepared by dissolving 1,3,5-trioxane (52.94 gms) in dichloromethane (150 ml)] to the reaction mixture at −20 to −15° C. Again Titanium tetrachloride solution [prepared by adding titanium tetrachloride (40 ml) and dichloromethane (50 ml)] was added to the reaction mixture at −20 to −15° C. Slowly raised the temperature of the reaction mixture to −13 to −8 ° C. and stirred for 45 minutes at the same temperature. Quenched the reaction mixture into pre-cooled 10% ammonium chloride solution at 0 to 5° C. Both the organic and aqueous layers were separated and extracted the aqueous layer with dichloromethane. Combined the organic layers and washed with water and followed by 20% sodium chloride solution. A solution of iodine (177.5 gms) and tetrahydrofuran (250 ml) was added to the organic layer at 10-15° C. and stirred for 1 hour at the same temperature. Reaction mixture was added to 30% sodium thiosulphate solution [prepared by using sodium thiosulphate (300 gms) and water (700 ml) and then the solution is washed with dichloromethane (75 ml)]. Both the organic layer and aqueous layers were separated and organic layer was washed with aqueous sodium thiosulphate solution. Distilled off the solvent from the organic layer at 40-45° C. and then co-distilled with methyl tertiary butyl ether. To the obtained compound, methyl tertiary butyl ether (500 ml) was added at 25-30° C. Heated the reaction mixture to 50-55° C. and stirred to get clear solution. Slowly cooled the reaction mixture to 30-35° C. and then further cooled to −5 to −8° C. and stirred for 2 hours at the same temperature. Filtered the precipitated solid, washed with methyl tertiary butyl ether. To the obtained wet compound methyl tertiary butyl ether (400 ml) was added at 25-30° C. Heated the reaction mixture to 50-55° C. and stirred to get clear solution. Slowly cooled the reaction mixture to 25-30° C. and then further cooled to −5 to −8° C. and stirred for 2 hours at the same temperature. Filtered the precipitated solid, washed with methyl tertiary butyl ether and dried to get the title compound. Yield: 95 gms.

### Example-15

### Preparation of 1-(((2R,4R)-2-(2,4-difluorophenyl)-4-(trityloxymethyl)tetrahydrofuran-2-yl)methyl)-1H-1,2,4-triazole (Formula-11)

A mixture of 1H-1,2,4-triazole (72.3 gm) and dimethylaminopyridine (5 gm) were added to dimethyl formamide (1250 ml) at 25-30° C. under nitrogen atmosphere. Cooled the reaction mixture to 5-10° C. Sodium tertiary butoxide (100.7 gm) was added to the reaction mixture in lot wise at 5-10° C. Raised the temperature of the reaction mixture to 25-30° C. and stirred for 2 hours at the same temperature. (2R,4R)-2-(2,4-difluorophenyl)-2-(iodomethyl)-4-(trityloxymethyl)tetrahydrofuran (250 gm) was added to the reaction mixture at 25-30° C. Heated the reaction mixture to 115-120° C. and stirred for 15 hours at the same temperature. Cooled the reaction mixture to 25-30° C. Water was slowly added to the reaction mixture at 25-30° C. and stirred for 2 hours at the same temperature. Filtered the precipitated solid, washed with water and dried to get the title compound.

### Example-16

### Purification of 1-(((2R,4R)-2-(2,4-difluorophenyl)-4-(trityloxymethyl)tetrahydrofuran-2-yl)-methyl)-1H-1,2,4-triazole (Formula-11)

Acetone (330 ml) was added to the compound obtained in above example-15 at 25-30° C. Heated the reaction mixture to 53-56° C. and stirred for 45 minutes at the same temperature. Cooled the reaction mixture to 25-30° C. and stirred for 45 minutes at the same temperature. Further, cooled the reaction mixture to 0-5° C. and stirred for 2 hours at the same temperature. Filtered the precipitated solid, washed with acetone and dried to get the title compound.

Yield: 166 gm; M.R: 150-156° C.

### Example-17

### Preparation of ((38,5R)-54(1H-1,2,4-triazol-1-yl)methyl)-5-(2,4-difluoro phenyl)tetrahydrofuran-3-yl)methyl 4-methylbenzenesulfonate (Formula-1)

Dichloromethane (1000 ml) was added to ((3R,5R)-5-((1H-1,2,4-triazol-1-yl)methyl)-5-(2,4-difluorophenyl)tetrahydrofuran-3-yl)methanol (135 gms) at 25-30° C. under nitrogen atmosphere. Cooled the reaction mixture to 10-15° C. P-toluene sulfonyl chloride (110 gms) and then followed by triethyl amine (118 gms) was slowly added to the reaction mixture at 10-15° C. Raised the temperature of the reaction mixture to 25-30° C. and stirred for 6 hours at the same temperature. After completion of the reaction, water was added to the reaction mixture. Both the organic and aqueous layers were separated and distilled off the solvent completely from the reaction mixture and then co-distilled with isopropanol at below 50° C. Isolated the obtained compound with isopropanol and dried the obtained compound to get title compound.

Yield: 188 gms.

### Example-18

### Preparation of ((3S,5R)-54(1H-1,2,4-triazol-1-yl)methyl)-5-(2,4-difluorophenyl)tetrahydrofuran-3-yl)methyl 4-methylbenzenesulfonate (Formula-1)

Dichloromethane (1000 ml) was added to ((3R,5R)-5-((1H-1,2,4-triazol-1-yl)methyl)-5-(2,4-difluorophenyl)tetrahydrofuran-3-yl)methanol (135 gms) at 25-30° C. under nitrogen atmosphere. Cooled the reaction mixture to 10-15° C. P-toluene sulfonyl chloride (110 gms) and then followed by triethyl amine (118 gms) was slowly added to the reaction mixture at 10-15° C. Raised the temperature of the reaction mixture to 25-30° C. and stirred for 6 hours at the same temperature. After completion of the reaction, water was added to the reaction mixture. Both the organic and aqueous layers were separated and distilled off the solvent completely from the reaction mixture and then co-distilled with isopropanol at below 50° C. Isolated the obtained compound with isopropanol and dried the obtained compound to get title compound.

Yield: 188 gms.

### Example-19

### Preparation of (3S,5R)-5-(2,4-difluorophenyl)-5-(iodomethyl)tetrahydrofuran-3-carboxylic acid

A mixture of sodium hydroxide (13.6 gms) and water (100 ml) was cooled to 5-10° C. Hydrogen peroxide (23.2 gms) was added to the above solution at 5-10° C. (R)-3-((3S,5R)-5-(2,4-di fluorophenyl)-5-(iodomethyl)tetrahydrofuran-3-carbonyl)-4-phenyloxazolidin-2-one (100 gms) and tetrahydrofuran (250 ml) were slowly added to the above reaction mixture at 5-10° C. and stirred for 3 hours at the same temperature. Sodium sulphite solution [prepared by adding sodium sulphite (20 gms) to water (200 ml)] was added to the reaction mixture at below 10° C. Water was added to the reaction mixture. Raised the temperature of the reaction to 25-30° C. and washed with dichloromethane. Adjusted the pH of the reaction mixture to 7.0 using 20% hydrochloric acid solution. Distilled off tetrahydrofuran from the reaction mixture under vacuum below 54° C. Adjusted the pH of the reaction mixture to 2.5 using 20% hydrochloric acid solution. Stirred the reaction mixture for 2 hours at 25-30° C. Cooled the reaction mixture to 5-10° C. and stirred for 2 hours at the same temperature. Filtered the precipitated solid, washed with purified water and dried to get the titlw compound.

Yield: 65 gms; Melting point: 100.8-102.8° C.

### Example-20

### Preparation of 4-(4-(4-(4-(((3R,5R)-5-(1H-1,2,4-triazol-1-yl)methyl)-5-(2,4-difluorophenyl)tetrahydrofuran-3-yl)methoxy)phenyl)piperazin-1-yl)phenyl)-1-((2S,3S)-2-(benzyloxy)pentan-3-yl)-1H-1,2,4-triazol-5(4H)-one

Sodium hydroxide solution [prepared by dissolving sodium hydroxide (7.8 gms) in water (10 ml)] was added to dimethyl sulfoxide (175 ml) at 25-30° C. ((3S,5R)-5-((1H-1,2,4-triazol-1-yl)methyl)-5-(2,4-difluorophenyl)tetrahydrofuran-3-yl)methyl-4-methylbenzene sulfonate (48.12 gms) and then followed by 1(2S,3S)-2-(benzyloxy)pentan-3-yl)-4-(4-(4-(4-hydroxyphenyl)piperazin-1 -yl)phenyl)-1H-1,2,4-triazol-5(4H)-one (50.0 gms) was added to the reaction mixture at 25-30° C. Heated the reaction mixture to 38-43° C. and stirred for 10 hours at the same temperature. Cooled the reaction mixture to 25-30° C. and slowly added to water at the same the same temperature. Cooled the reaction mixture to 10-15° C. and adjusted the pH of the reaction mixture to 7.0 using hydrochloric acid solution at the same temperature. Raised the temperature of the reaction mixture to 25-30° C. and stirred for 3 hours at the same temperature. Filtered the precipitated solid and washed with purified water. To the obtained wet compound, isopropanol (500 ml) was added at 25-30° C. Heated the reaction mixture to 65-70° C. to get clear solution. Cooled the reaction mixture to 25-30° C. and stirred for 4 hours at the same temperature. Filtered the precipitated solid, washed with isopropanol and dried to get the title compound.

Yield: 69.29 gms.

### Example-21

### Preparation of 4-(4-(4-(4-(((3R,5R)-5-(1H-1,2,4-triazol-1-yl)methyl)-5-(2,4-difluorophenyl)tetrahydrofuran-3-yl)methoxy)phenyl)piperazin-1-yl)phenyl)-1-((2S,3S)-2-hydroxypentan-3-yl)-1H-1,2,4-triazol-5(4H)-one (Posaconazole)

4-(4-(4-(4-(((3R,5R)-5-((1H-1,2,4-triazol-1-yl)methyl)-5-(2,4-difluorophenyl)tetrahydrofuran-3-yl)methoxy)phenyl)piperazin-1-yl)phenyl)-1-((2S,3S)-2-(benzyloxy)pentan-3-yl)-1H-1,2,4-triazol-5 (4H)-one (50 gms) was added to hydrochloric acid (150 ml) at 25-30° C. Heated the reaction mixture to 60-63° C. and stirred for 11 hours at the same temperature. A mixture of water (500 ml), methanol (400 ml) and acetone (200 ml) was added to the reaction mixture at 25-30° C. Cooled the reaction mixture to 10-15° C. Adjusted the pH of the reaction mixture to 7.0 using aqueous sodium hydroxide solution below 15° C. Raised the temperature of the reaction mixture to 25-30° C. and stirred for 1 hour at the same temperature. Filtered the precipitated solid, washed with water. To the obtained wet compound, acetone (500 ml) was added at 25-30° C. Heated the reaction mixture to 55-60° C. and stirred for 30 minutes at the same temperature. Carbon (5.0 gms) was added to the reaction mixture at 55-60° C. and stirred for 30 minutes at the same temperature. Filtered the reaction mixture through by-flow bed and washed with hot acetone. Distilled off the solvent from the filtrate under reduced pressure. Acetone (500 ml) was added to the obtained compound at 25-30° C. Heated the reaction mixture to 55-60° C. and stirred for 30 minutes at the same temperature. Cooled the reaction mixture to 25-30° C. Water was slowly added to the reaction mixture at 25-30° C. and stirred for 2 hours at the same temperature. Filtered the precipitated solid, washed with water and dried to get the title compound.

Yield: 41 gms.

### Example-22

### Purification of 4-(4-(4-(4-(((3R5R)-5-(1H-1,2,4-triazol-1-yl)methyl)-5-(2,4-difluorophenyl)tetrahydrofuran-3-yl)methoxy)phenyl)piperazin-1-yl)phenyl)-1-((2S,3S)-2-hydroxypentan-3-yl)-1H-1,2,4-triazol-5(4H)-one (Posaconazole)

Isopropanol (700 ml) was added to the 4-(4-(4-(4-(((3R,5R)-5-((1H-1,2,4-hiazol-1-yl)methyl)-5-(2,4-difluorophenyl)tetrahydrofiiran-3-yl)methoxy)phenyl)piperazin-1-yl) phenyl)-1-(2S,3S)-2-hydroxypentan-3-yl)-1H-1,2,4-triazol-5 (4H)-one obtained in the above example-8 at 25-30° C. Heated the reaction mixture to 80-85° C. and stirred for 45 minutes at the same temperature. Cooled the reaction mixture to 25-30° C. and stirred for 5 hours at the same temperature. Filtered the precipitated solid, washed with isopropanol and dried to get the title compound.

Yield: 41 gms; Melting point: 168-171° C.

### Example-23

### Preparation of amorphous Posaconazole Compound of Formula-I:

Dissolved posaconazole (50 g) in dichloromethane (250 ml) at 25-35° C. and the resulting mixture was stirred to get clear solution. The solution was filtered and washed with dichloromethane (50 ml). The filtrate was slowly added to methyl tertiary butyl ether [MTBE] (2000 ml) at −30 to −20° C. for a period of 2 hrs. The resulting mixture was stirred for 1½ hrs at the same temperature. The precipitated compound was filtered and washed with MTBE and dried to get the title compound.

Yield: 96.8%. Water content: 0.65% w/w; Purity by HPLC: 99.77%; Chiral purity: 99.71% w/w by HPLC.

Particle size distribution: D(0.9): 199.84 μm; D(4,3): 75.323 μm;

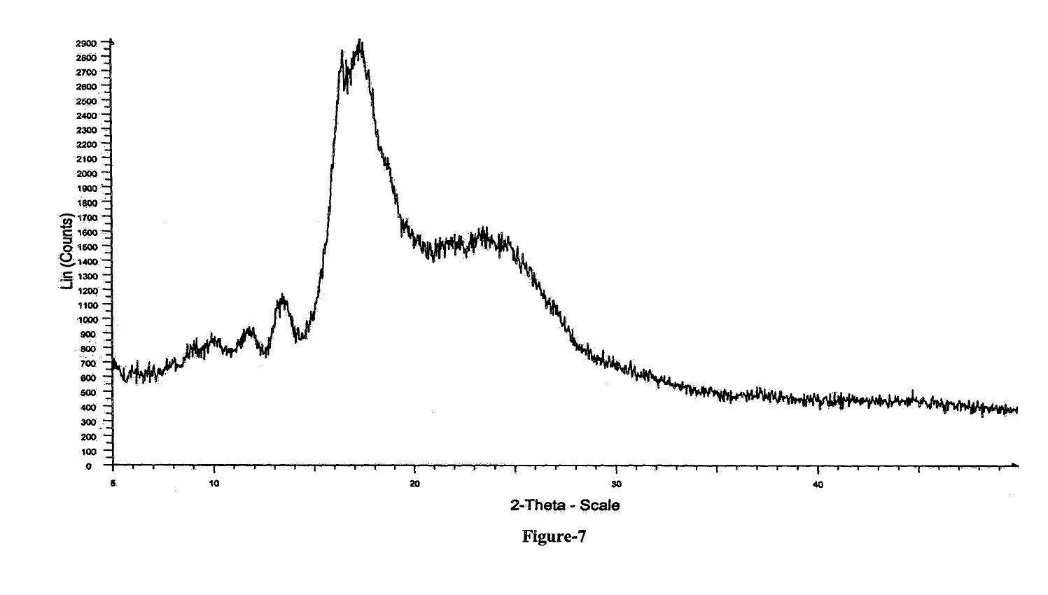

The P-XRD pattern of amorphous Posaconazole was shown in FIG. 7.

### Example-24

### Preparation of amorphous Posaconazole Compound of Formula-1

The above example can be repeated by using n-heptane in place of methyl tertiary butyl ether to get the amorphous Posaconazole.

1-23. (canceled)

24. A process for the preparation of ((3S,5S)-5-(1H-1,2,4-triazol-1-yl)methyl)-5-(2,4-difluorophenyl)tetrahydrofuran-3-yl)methyl-4-methyl benzertesulfonate compound of formula-1 comprising:
a) reacting 4-(2,4-difluorophenyl)-4-oxobutatioic acid compound of formula-2 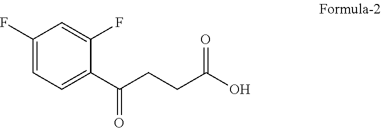 [SMILES: O=C(O)CCC(=O)c1ccc(F)cc1F]
with methyl triphenyl phosphonium bromide in the presence of a suitable base in a suitable solvent to provide 4-(2,4-difluorophenyl)pent-4-enoic acid compound of formula-3; 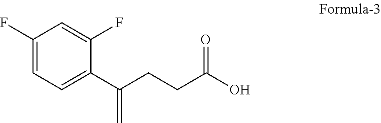
b) reacting the compound of formula-3 with (R)-4-phenyloxazolidin-2-one compound of formula-4 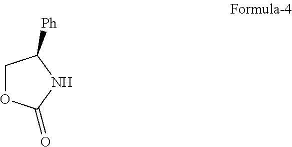 [SMILES: O=C1N[C@H](c2ccccc2)CO1]
in the presence of suitable coupling agent and a suitable base in a suitable solvent to provide (R)-3-(4-(2,4-difluorophenyl)pent-4-enoyl)-4-phenyloxazolidin-2-one compound of formula-5: 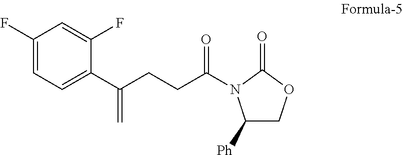 [SMILES: C=C(CCC(=O)N1C(=O)OC[C@H]1c1ccccc1)c1ccc(F)cc1F]
c) reacting the compound of formula-5 with 1,3,5-trioxane in the presence of a suitable base and suitable catalyst in a suitable solvent to provide (R)-3-((S)-4-(2,4-difluorophenyl)-2-(hydroxymethyl)pent-4-enoyl)-4-phenyloxarzolidin-2-one compound of formula-6; 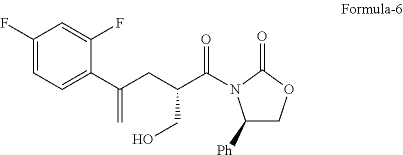 [SMILES: C=C(C[C@@H](CO)C(=O)N1C(=O)OC[C@H]1c1ccccc1)c1ccc(F)cc1F]
d) cyclizing the compound of formula-6 in-situ in the presence of iodine and a suitable base in a suitable solvent to provide (R)-3(3S,5R)-5-(2,4-difluorophenyl)-5-(iodomethyl)tetrahydrofuran-3-carbonyl)-4-phenyloxazolidin-2-one compound of formula-7; 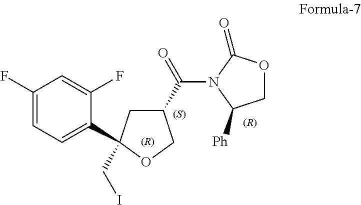
e) hydrolyzing the compound of formula-7 in the presence of a base and a suitable catalyst in a suitable solvent to provide (3S,5R)-5-(2,41-difluorophenyl)-5-(iodomethyl)tetrahydrofuran-3-carboxylic acid compound of formula-8; 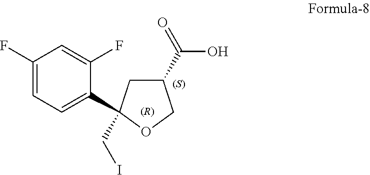
f) reducing the compound of formula-8 with a suitable reducing agent in a suitable solvent to provide (3R,5R)-5-(2,4-dilluorophenyl)-5-(iodomethyl)tetrahydrofuran-3-yl)methanol compound of formula-9; 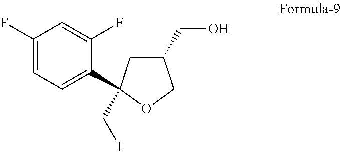 [SMILES: OC[C@@H]1CO[C@@](CI)(c2ccc(F)cc2F)C1]
g) reacting the compound of formula-9 with trityl chloride in the presence of a suitable base in a suitable solvent to provide (2R,4R)-2-(2,4-dithiorophenyl)-2-(iodomethyl)-4-(trityloxy methyl)tetrahydroaran compound of formula-10; 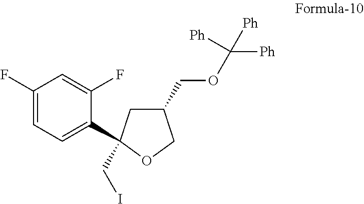 [SMILES: Fc1ccc([C@@]2(CI)C[C@H](COC(c3ccccc3)(c3ccccc3)c3ccccc3)CO2)c(F)c1]
h) reacting the compound of formula-10 with 1H-1,2,4-triazole in the presence of a suitable base in a suitable solvent to provide 1-(((2R,4R)-2-(2,4-difluorophenyl)-4-(trityloxy methyl)tetrahydrofuran-2-yl)methyl)-1H-1,2,4-triazole compound of formula-11; 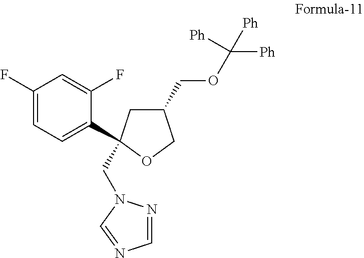 [SMILES: Fc1ccc([C@@]2(Cn3cncn3)C[C@H](COC(c3ccccc3)(c3ccccc3)c3ccccc3)CO2)c(F)c1]
i) reacting the compound of formula-11 with a suitable deprotecting agent in a suitable solvent to provide ((3R,5R.)-5-((1H-1,2,4-triazol-1-yl)methyl)-5-(2,4-difluorophenyl)tetrahydrofuran-3-yl) methanol compound of formula-12; and 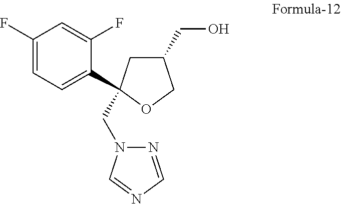 [SMILES: OC[C@@H]1CO[C@@](Cn2cncn2)(c2ccc(F)cc2F)C1]
j) reacting the compound of formula-12 with p-toluenesulfonyl chloride in the presence of base in a suitable solvent to provide (3S,5R)-5-((1H-1,2,4-triazol-1-yl)methyl)-5-(2,4-difluorophenyl)tetrahydrofuran-3-yl)methyl 4-methylbenzenesulfonate compound of formula-I.

25. The process according to claim 24, wherein
in step a) the suitable base is selected from an organic or an inorganic base; and the suitable solvent is selected from the group consisting of chloro solvents, ester solvents, ketone solvents, hydrocarbon solvents, polar aprotic solvents, and alcohol solvents, or a mixture thereof;
in step b) the suitable coupling agent is selected from the group consisting of dicyclohexylcarbodiimide (DCC), diisopropylcarbodlimide (DIC), dimethylamino)propylcarbodiimide hydrochloride (EDC), and N,N-carbonyldiimidazole (CDI); the suitable base is selected from an organic or an inorganic base; and the suitable solvent is selected from the group consisting of chloro solvents, ester solvents, ketone solvents, hydrocarbon solvents, polar aprotic solvents, and alcohol solvents, or a mixture thereof;
in step c) the suitable base is an organic base selected from the group consisting of triethylaniine, tributyl amine, pyridine, 4-dimethylaminopyridint, N-methyl morpholine and diisopropylethyl amine; the suitable solvent is selected from the group consisting of alcohol solvents, chloro solvents, ketone solvents, ether solvents, and ester solvents, or a mixture thereof; and the suitable catalyst is TiCl₄.
in step d) the suitable base is an inorganic base selected from the group consisting of alkali metal hydroxides, alkali metal alkoxides, alkali metal carbonates, and alkali metal bicarbonates; and the suitable solvent is selected from the group consisting of chloro solvents, ether solvents, alcoholic solvents, ester solvents, hydrocarbon solvents, and ketone solvents, or a mixture thereof;
in step e) the suitable catalyst is hydrogen peroxide; the suitable base is an inorganic base selected from the group consisting of alkali metal hydroxides, alkali metal alkoxides, alkali metal carbonates, and alkali metal bicarbonates; and the suitable solvent is selected from the group consisting of ether solvents, alcohol solvents, ester solvents, ketone solvents, and hydrocarbon solvents, or a mixture thereof;
in step f) the suitable reducing agent is selected from the group consisting of DIBAL-H, lithium aluminium hydride, sodium borohydride, lithium borohydride, NaBH₃CN, sodium borohydride/BF₃-etherate, vitride, sodium borohydride/aluminium chloride or borane/aluminium chloride, and sodium borohydride/iodine; and the suitable solvent is selected from the group consisting of ether solvents, chloro solvents, ester solvents, hydrocarbon solvents, ketone solvents, and alcohol solvents, or a mixture thereof;
in step g) the suitable base is an organic base selected from the group consisting of triethylamine, tributyl amine, pyridine, 4-dimethylaminopyridine, N-methyl morpholine and diisopropylethyl amine; and the suitable solvent is selected from alcohol solvents, chloro solvents, ketone solvents, ether solvents, and ester solvents, or a mixture thereof;
in step h) the suitable base is selected from an organic or inorganic base, wherein the organic base is selected from the group consisting of triethylamine, tributyl amine, pyridine, 4-dimethyl aminopyridine, N-methyl morpholine and diisopropylethyl amine, and the inorganic base is selected from the group consisting of alkali metal hydroxides, alkali metal alkoxides, alkali metal carbonates, and alkali metal bicarbonates; and the suitable solvent is selected from the group consisting of polar aprotic solvents, chloro solvents, alcoholic solvents ester solvents, and hydrocarbon solvents, or a mixture thereof;
in step i) the suitable deprotecting agent is a mineral acid selected from the group consisting of sulfuric acid, hydrochloric acid, nitric acid, phosphoric acid, boric acid, hydrofluoric acid, hydrobromic acid, and perchloric acid; the suitable solvent is selected from the group consisting of chloro solvents, ester solvents, ketone solvents, alcohol solvents, ether solvents, polar solvents, and polar aprotic solvents, or a mixture thereof; and
in step j) the suitable base is an organic base selected from the group consisting of triethylamine, tributyl amine, pyridine, 4-dimethylaminopyridine, N-methyl morpholine and diisopropylethyl amine; and the suitable solvent is selected from the group consisting of chloro solvents, ester solvents, ketone solvents, alcoholic solvents, and hydrocarbon solvents, or a mixture thereof.

26. The process according to claim 24, wherein the process for the preparation of ((3S,5R)-5-((1H-1,2,4-triazol-1-yl)methyl)-5-(2,4-difluorophenyl)tetrahydrofuran-3-yl)methyl-4-methyl benzenesulfonate compound of formula-1 comprises:
a) reacting 4-(2,4-difluorophenyl)-4-oxobutanoic acid compound of formula-2 with methyl triphenyl phosphonium bromide in the presence of sodium tertiary butoxide in dimethyl sulfoxide to provide 4-(2,4-difluorophenyl)pent-4-enoic acid compound of formula-3;
b) reacting the compound of formula-3 with (R)-4-phenyloxazolidin-2-one compound of formula-4 in the presence of dicyclohexyl carbodiimide and dimethyl amino pyridine in dichloromethane to provide (R)-3-(4-(2,4-difluorophenyl)pent-4-enoyl)-4-phenyloxazolidin-2-one compound of formula-5;
c) reacting the compound of formula-5 with 1,3,5-trioxane in the presence of diisopropyl ethyl amine and titanium tetra chloride in dichloromethane to provide (R)-3-((S)-4-(2,4-difluorophenyl)-2-(hydroxymethyl)pent-4-enoyl)-4-phenyloxazolidin-2-one compound of formula-6;
d) cyclizing the compound of formula-6 in-situ in the presence of iodine and sodium carbonate in dichloromethane to provide (R)-3-(3S,5R)-5-(2,4-difluorophenyl)-5-(iodomethyl)tetrahydrofuran-3-carbonyl)-4-phenyloxazolidin-2-one compound of formula-7;
e) hydrolyzing the compound of formula-7 in the presence of sodium hydroxide and hydrogen peroxide in tetrahydrofuran to provide (3S,5R)-5-(2,4-difluorophenyl)-5-(iodomethyl)tetrahydrofuran-3-carboxylic acid compound of formula-8;
f) reducing the compound of formula-8 with sodium horohydride in BF3-etherate in tetrahydrofuran to provide (3R,5R)-5-(2,4-difluorophenyl)-5-(iodomethyl)tetrahydrofuran-3-yl )methanol compound of formula-9;
g) reacting the compound of formula-9 with trityl chloride in presence of triethyl amine in dichloromethane to provide (2R,4R)-2-(2,4-difluorophenyl)-2-(iodomethyl)-4-(trityloxymethyl)tetrahydrofuran compound of formula-10;
h) reacting the compound of formula-10 with 1H-1,2,4-triazole in the presence of sodium tertiary butoxide and dimethylamino pyridine in dimethylformamide to provide 1-(((2R,4R)-2-(2,4-difluorophenyl)-4-(trityloxymethyl)tetrahydrofuran-2-yl)methyl)-1H-1,2,4-triazole compound of formula-11;
i) reacting the compound of formula-11 with sulfuric acid in acetone to provide ((3R,5R)-5-((1H-1,2,4-triazol-1-yl)methyl)-5-(2,4-difluorophenyl)tetrahydrofuran-3-yl)methanol compound of formula-12;
j) reacting the compound of formula-12 with p-toluenesulfonyl chloride in the presence of dimethylamino pyridine in dichloromethane to provide compound of formula-1.

27. The process according to claim 24, wherein the process for the preparation of ((3S,5R)-5-((1H-1,2,4-triazol-1-yl)methyl)-5-(2,4-difluorophenyl) tetrahydrofuran-3-yl)methyl-4-methylbenzenesulfonate compound of formula -1 comprises:
a) reacting (3R,5R)-5-(2,4-difluorophenyl)-5-(iodomethyl)tetrahydrofuran-3-yl)methanol compound of formula-9 with trityl chloride in the presence of triethyl amine in dichloromethane to provide (2R,4R)-2-(2,4-difluorophenyl)-2-(iodomethyl)-4-(trityloxy methyl)tetrahydrofuran compound of formula-10;
b) reacting the compound of formula-10 with 1H-1,2,4-triazole in the presence of sodium tertiary butoxide and dimethylamino pyridine in dimethylformamide to provide 1-(2R,4R)-2-(2,4-difluorophenyl)-4-(trityloxymethyl)tetrahydrofuran-2-yl)methyl)-1H-2,4-triazole compound of formula-11;
c) reacting the compound of formula-11 with hydrochloric acid in methanol to provide ((3R,5R)-5-((1H-1,2,4-triazol-1-yl)methyl)-5-(2,4-difluoropherryl)tetrahydrofuran-3-yl)methanol compound of formula-12; and
d) reacting the compound of formula-12 with p-toluenesulfonyl chloride in the presence of dimethylamino pyridine in dichloromethane to provide compound of formula-1.

28. The process according to claim 24, wherein the process for the preparation of (R)-3-(4-(2,4-difluorophenyl)pent-4-enoyl)-4-phenyloxazolidin-2-one compound of formula-5, comprises:
reacting 4-(2,4-difinoroplienyl)pent-4-enoic acid compound of formula-3 with (R)-4-phenyloxazolidin-2-one compound of formula-4 in the presence of a suitable coupling agent and in presence of a suitable base in a suitable solvent.

29. The process according to claim 28, wherein the suitable coupling agent is selected from the group consisting of dicyclohexylcarbodiimide (DCC), diisopropylcarbodiimide (DIC), ethyl-(N″,N″-dimethyl amino) propylcarbodiimide hydrochloride (EDC), and N,N-carbonyldiimidazole (CDI); the suitable base is selected from an organic or inorganic base; and the suitable solvent is selected from the group consisting of chloro solvents, ester solvents, ketone solvents, hydrocarbon solvents, polar aprotic solvents, and alcohol solvents, or mixture thereof.

30. The process according to claim 28, wherein the process for the preparation of (R)-3-(4-(2,4-difluorophenyl)pent-4-enoyl)-4-phenyloxazolidin-2-one compound of formula-5, comprises:
reacting 4-(2,4-difluorophenyl)pent-4-enoic acid compound of formula-3 with (R)-4-phenyloxazolidin-2-one compound of formula-4 in presence of dicyclohexylcarbodiimide and dimethyl amino pyridine in dichloromethane.

31. Compounds having the following structural formulae:

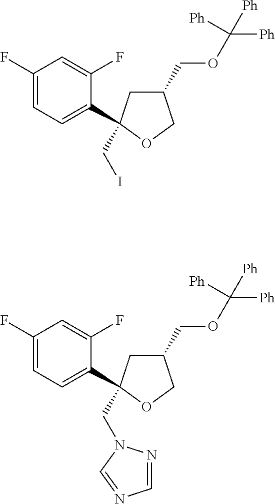 [SMILES: Fc1ccc([C@@]2(CI)C[C@H](COC(c3ccccc3)(c3ccccc3)c3ccccc3)CO2)c(F)c1 ; Fc1ccc([C@@]2(Cn3cncn3)C[C@H](COC(c3ccccc3)(c3ccccc3)c3ccccc3)CO2)c(F)c1]

32. The compounds according to claim 31, wherein the (2R,4R)-2-(2,4-difluorophenyl)-2-(iodomethyl)-4-(trityloxymethyl)tetrahydrofuran (Crystalline form-M) is acterized by:
a) having peaks at 4.3, 8.8, 10.1, 11.8, 12.1, 12.6, 13.2, 14.7, 15.0, 15.6, 16.7, 16.9, 18.2, 18.6, 18.9, 19.2, 19.4, 21.7, 22.1, 23.2, 23.5, 24.2, 24.5, 25.1, 25.5, 26.0, 26.6, 27.1, 29.0, 30.2, 30.9, 31.6, 32.4. 32.7, 33.2, 34.6, 35.8 and 43.5±0.2 degrees of two-theta in a powder X-ray diffractogram; and/or
b) its DSC thermogram showing sharp endotherm at 141.00° C. as illustrated in FIG. 2; and/or
c) its IR spectrum having absorption bands as illustrated in FIG. 3.

33. The compounds according to claim 31, wherein the 1-(((2R,4R)-2-(2)4-difluorophenyl)-4-(tritloxmethyl)tetrahydrofuran-2-yl)methyl)-1H-1,2,4-triazole (Crystalline form-S) is characterized by:
a) having peaks at 4.5, 9.0, 9.4, 9.8, 10.9, 12.1, 13.9, 14.7 15.2, 15.5, 15.9, 18.8, 19.4 20.0, 21.1, 21.8, 22.2, 23.0, 24.5, 24.8, 27.8 and 30.0±0.2 degrees of two-theta in a powder X-ray diffractogram; and/or
b) its DSC thermogram showing sharp endotherm at 154.58° C. as illustrated in FIG. 5; and/or
c) its IR spectrum having absorption bands as illustrated in FIG. 6.

34. The process according to claim 24, wherein the ((3R,5R)-5(1H-1,2,4-triazol-1-yl)methyl)-5-(2,4-difluorophenyl)tetrahydrofuran-3-yl)methanol compound of formula-12 is substantially free of cyclized impurity.

35. The process according to claim 24, wherein the process for the preparation of (R)-3((3S,5R)-5-(2,4-difluorophenyl)-5-(iodomethyl)tetrahydrofuran-3-carbonyl)-4-phenyloxazolidin-2-one compound of formula-7 comprises:
a) reacting the compound of formula-5 with 1,3,5-trioxane in the presence of a suitable base and suitable catalyst in a suitable solvent to provide (R)-3-((S)-4-(2,4-difluorophenyl)-2-(hydroxymethyl)pent-4-enoyl)-4-phenyloxazolidin-2-one compound of formula-6; and
b) cyclizing the compound of formula-6 in-situ in the presence of iodine, in absence of a base, in a suitable solvent to provide (R)-3-(3S,5R)-5-(2,4-difluorophenyl)-5-(iodomethyl)tetrahydrofuran-3-carbonyl)-4-phenyloxazolidin-2-one compound of formula-7.

36. The process according to claim 35, wherein the process for the preparation of (R)-3-(3S,5R)-5-(2,4-difluorophanyl)-5-(iodomethyl)tetrahydrofuran-3-carbonyl)-4-phenyloxazolidin-2one compound of formula-7 comprises:
a) reacting the compound of formula-5 with 1,3,5-trioxane in the presence of diisopropyl ethyl amine and titanium tetrachloride in dichloromethane to provide (R)-3-((S)-4-(2,4-difluorophenyl)-2-(hydroxymethyl)pent4-enoyl)-4-phenyloxazolidin-2-one compound of formula-6; and
b) cyclizing the compound of formula-6 in-situ in the presence of iodine in dichloromethane to provide (R)-3-((3S,5R)-5-(2,4-difluorophenyl)-5-(iodomethyl)tetrahydrofuran-3-carbonyl)-4-phenyloxazolidin-2-one compound of formula-7.

37. The process according to claim 24, wherein the process for the purification of 1-(((2R,4R)-2-(2,4-difluorophenyl)-4-(trityloxymethyl)tetrahydrofuran-2-yl)methyl)-1H-1,2,4-triazole compound of formula-11 comprises:
a) dissolving 1-(((2R,4R)-2-(2,4-difluorophenyl)-4-(trityloxymethyl)tetrahydrofuran-2-yl)methyl)-1H-1,2,4-triazole compound of formula-11 in a suitable solvent to form a reaction mixture;
b) heating the reaction mixture;
c) cooling the reaction mixture; and
d) filtering and drying the solid to get pure 1-(((2R,4R)-2-(2,4-difluorophenyl)-4-(trityloxymethyl)tetrahydrofuran-2-yl)methyl)-1H-1,2,4-triazole compound of formula-11.

38. The process according to claim 37, wherein the process for the purification of 1-(((2R,4R)-2-(2,4-difluorophenyl)-4-(trityloxymethyl)tetrahydrofuran-2-yl)methyl)-1H-1,2,4-triazole compound of formula-11 comprises:
a) dissolving 1-(((2R,4R)-2-(2,4-difluorophenyl)-4-(trityloxymethyl)tetrahydrofuran-2-yl)methyl)-1H-1,2,4-triazole compound of formula-11 in acetone to form a reaction mixture;
b) heating the reaction mixture;
c) cooling the reaction mixture; and
d) filtering and drying the solid to get pure 1-(((2R,4R)-2-(2,4-difluorophenyl)-4-(trityloxymethyl)tetrahydrofuran-2-yl)methyl)-1H-1,2,4-triazole compound of formula-11.

39. A process for the preparation of amorphous 4-[4-[4-[4-[[(3R,5R)-5-(2,4-difluorophenyl)tetrahydro-5-(1H-1,2,4-triazol-1-methyl)-3furanyl]methoxyl]phenyl]-1-piperazinyl]phenyl]-2-[(1S,2S)-1-ethyl-2-hydroxypropyl]-2,4-dihydro-3H-1,2,4-triazol3-one compound of formula-I; 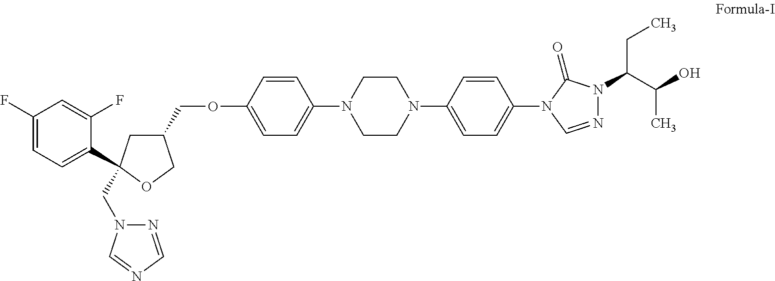 [SMILES: CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O]
the process comprising:
a) dissolving the compound of formula-I in a suitable solvent to form a reaction mixture;
b) stirring the reaction mixture;
c) filtering the reaction mixture;
d) adding filtrate to a suitable anti-solvent;
e) stirring the reaction mixture; and
f ) filtering the solid and then drying to get amorphous form of compound of formula-I.

40. The process according to claim 39, wherein the suitable solvent used in step-a) is selected from the group consisting of chloro solvents, ketone solvents, ester solvents, and alcohol solvents or a mixture thereof.

41. The process according to claim 39, wherein the suitable anti-solvent used in step-d) is selected from ether solvents or hydrocarbon solvents.

42. The process according to claim 39, wherein the process for the preparation of amorphous 4-[4-[4-[4-[[(3R,5R)-5-(2,4-difluorophenyl)tetrahydro-5-(1H-1,2,4-triazol-1-ylmethyl)-3furanyl]methoxy]phenyl]-1-piperazinyl]phenyl]-2-[(1S,2S)-1-ethyl-2-hydroxypropyl]-2,4-dihydro-3H-1,2,4-triazol-3-one compound of formula-I comprises:
a) dissolving the compound of formula-I in dichloromethane to form a reaction mixture;
b) stirring the reaction mixture;
c) filtering the reaction mixture;
d) adding filtrate to methyl tertiarybutyl ether;
e) stirring the reaction mixture; and
f ) filtering the solid and then drying to get amorphous form of compound of formula-I.

43. The process according to claim 24, wherein the (2R,4R)-2-(2,4-difluorophenyl)-2-(iodomethyl)-4-(trityloxymethyl)tetrahydrafuran compound of formula-10 and 1-(((2R,4R)-2-(2,4-difluorophenyl)-4-(trityloxymethyl)tetrahydrofuran-2-yl)methyl)-1H-1,2,4-triazole compound of formula-11 and their crystalline forms useful in the preparation of pure compound of formula-1.
